(19) United States
(12) Patent Application Publication
(10) Pub. No.: US 2010/0152206 A1
(43) Pub. Date: Jun. 17, 2010

(54) Bicyclic Dihydropyrimidines and Uses Thereof

(76) Inventors: Ralph Mazitschek, Arlington, MA (US); John A. Tallarico, Brookline, MA (US); Zoltan Maliga, East Brunswick, NJ (US); Timothy J. Mitchison, Brookline, MA (US)

Correspondence Address: Choate, Hall & Stewart;Patent Group, Two International Place, Boston, MA, 02110, US

(21) Appl. No.: 11/794,586

(22) PCT Filed: Jan. 6, 2006
(86) PCT No.: PCT/US06/00300

§ 371 Date: Oct. 5, 2009

Related U.S. Application Data
(60) Provisional application No. 60/642,360, filed on Jan. 7, 2005.

(51) Int. Cl. A61K 31/519; C07D 491/052; C07D 491/048; C07D 487/04; C07D 471/04; A61P 35/00; C12N 5/00

(52) U.S. Cl. 514/260.1; 544/278; 544/280; 544/279; 514/264.1; 514/265.1; 435/375

(57) ABSTRACT

The present invention provides compounds having formula (I): (I) and pharmaceutically acceptable derivatives thereof; as described generally and in subclasses herein, which compounds are useful as kinesin inhibitors (e.g., Eg5 inhibitors), and thus are useful, for example, for the treatment of proliferative disorders e.g., cancer. The invention additionally provides methods for preparing compounds of the invention, compositions comprising them, and methods for the use thereof in the treatment of various disorders where Eg5 is involved. In certain embodiments, the present invention provides for compounds, compositions, methods and systems for inhibiting cell growth. More specifically, the present invention provides for methods, compounds and compositions which are capable of inhibiting mitosis in metabolically active cells. Compounds, compositions and methods of the present invention inhibit the activity of a protein involved in the assembly and maintenance of the mitotic spindle. One class of proteins which acts on the mitotic spindle is the family of mitotic kinesins, a subset of the kinesin superfamily.

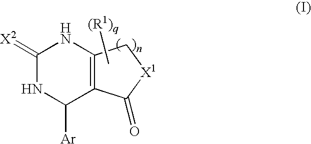

### PRIORITY

The present application claims priority to U.S. Provisional Patent Application No. 60/642,360 filed Jan. 7, 2005; The entire contents of which are incorporated herein by reference.

### GOVERNMENT SUPPORT

The work described in the present application was supported, in part, by grant number CA78048 from the National Cancer Institute, and grant number GM62566 from the National Institute of General Medical Sciences. The U.S. government may have certain rights in this invention.

### BACKGROUND OF THE INVENTION

Cell-permeable small molecules can rapidly perturb the function of their targets and are therefore powerful tools to dissect dynamic cellular processes. However, such modulators are not available for most of the proteins involved in essential processes, and many of the ones that are available are nonspecific. The only known small molecules that specifically affect the mitotic machinery target tubulin (E. Hamel, Med. Res. Rev. 16, 207 (1996)), a subunit of the microtubules in the mitotic spindle.

In all eukaryotic cells, the formation of a microtubule based dynamic structure, commonly known as the spindle, is essential for the accurate segregation of chromosomes during cell division (T. Wittmann, A. Hyman, A. Desai, Nat Cell Biol 2001, 3, E28-34). Many approved anti-cancer drugs such as taxanes (Taxol) or vinca alkaloids (Vinblastine) directly target the dynamic process of microtubule polymerization and depolymerization. However, microtubule based structures are also important for other cellular processes, including the organization of intracellular structures as well as intracellular transport, and perturbing these processes results in undesired side effects. Because the above-mentioned drugs do not selectively target the spindle-associated microtubules, the application of those compounds is severely limited (E. K. Rowinsky, Clin Cancer Res 1999, 5, 481-486).

One class of proteins involved in the assembly and maintenance of the mitotic spindle is the family of mitotic kinesins, a subset of the kinesin superfamily. This superfamily contains over 100 proteins, whose other functions include organelle transport and membrane organization (R. D. Vale and R. J. Fletterick, Annu. Rev. Cell Dev. Biol. 13, 745 (1997)). Interestingly, motor proteins involved in the formation of the spindle are generally not associated with other microtubule-based structures (1. T. Wittmann, A. Hyman, A. Desai, Nat Cell Biol 2001, 3, E28-34; 2. E. K. Rowinsky, Clin Cancer Res 1999, 5, 481-486; and 3. M. Schliwa, G. Woehlke, Nature 2003, 422, 759-765). The first evidence that mitotic kinesins are important in establishing spindle bipolarity came from genetic studies: temperature-sensitive mutants in the BimC family of kinesins do not form bipolar spindles at the restrictive temperature (A. P. Enos and N. R. Morris, Cell 60, 1019 (1990); I. Hagan and M. Yanagida, Nature 356, 74 (1992); M. A. Hoyt et al., J. Cell Biol. 118, 109 (1992)). Inhibition of the BimC kinesin Eg5 with Eg5-specific antibodies also induced monoasters similar to those observed after treatment with monastrol (A. Blangy et al., Cell 83, 1159 (1995); K. E. Sawin et al., Nature 359, 540 (1992)). Like other kinesins, Eg5 can drive the movement of microtubules in vitro (T. M. Kapoor and T. J. Mitchison, Proc. Natl. Acad. Sci. U.S.A. 96, 9106 (1999)).

Enzymes in the kinesin superfamily use the free energy of ATP hydrolysis to drive intracellular movement and influence cytoskeleton organization (R. D. Vale and R. J. Fletterick, Annu. Rev. Cell. Dev. Biol. 13, 745-777 (1997)). More than 90 members of this family are known. Historically, kinesins have been proposed to move cellular cargo along polar microtubule tracks. More recently it has been shown that these ATPases can modulate dynamics of the underlying microtubule network (A. Desai et al., Cell 96, 69-78 (1999)), couple movement of cargo to the microtubule polymerization or depolymerization (K. W. Wood et al., Cell 91, 357-366 (1997)), and crosslink microtubules in dynamic structures (D. J. Sharp et al., J. Cell Biol. 144, 125-138 (1999)). Kinesins thus play central roles in mitotic and meiotic spindle formation, chromosome alignment and separation, axonal transport, endocytosis, secretion, and membrane trafficking. The cargo associated with these motor proteins includes intracellular vesicles, organelles, chromosomes, kinetochores, intermediate filaments, microtubules, and even other motors (reviewed in C. E. Walczak and T. J. Mitchison, Cell 85, 943-946 (1996); and N. Hirokawa, Science 279, 519-526 (1998)).

For many of these processes, more than one kinesin is implicated, and the specific cargo associated with a given motor protein has been difficult to establish. For example, conventional kinesin (R. D. Vale et al., Cell 42, 39-50 (1985)) (the founding member of the family) is one of a subset of kinesins involved in organelle transport in mammalian cells. This group includes KIF1, KIF2, KIFC2/C3, and KIF4; and more recently, 18 new murine KIFs have been reported, many of which may functionally overlap with the transport kinesins (reviewed in N. Hirokawa, Science 279, 519-526 (1998)). It thus has been difficult to tie down the in vivo function(s) of conventional kinesin. Experiments using antisense techniques and microinjection of inhibitory antibodies have been further complicated by recent observations of efficient endoplasmic reticulum to Golgi transport in the absence of microtubules, albeit under restricted conditions (reviewed in G. S. Bloom and L. S. Goldstein, J. Cell Biol. 140, 1277-1280 (1998)). Similar problems have been encountered in dissecting the function of kinesins in mitosis. Extensive genetic analysis of motors in Saccharomyces cerevisiae has linked all but one of the six kinesins to spindle function. None of these five motors are individually required for the viability of yeast, implying that more than one motor is associated with essential aspects of spindle movement (W. S. Saunders and M. A. Hoyt, Cell 70, 451-458 (1992); M. A. Hoyt et al., Proc. Natl. Acad. Sci. USA 94, 12747-12748 (1997)) Immunodepletion and add-back approaches in Xenopus extract spindle assembly assays have provided similarly ambiguous data (C. E. Walczak et al., Curr. Biol. 8, 903-913 (1998)).

Small molecules that conditionally activate or inactivate a protein are valuable tools for analyzing cellular functions of proteins (D. T. Hung et al., Chem. Biol. 3, 623-639 (1996)). Their use provides an alternative to conventional biochemical and genetic approaches. However, to date there have been few reports of small molecules that can reversibly alter the function of motor proteins. Butanedione monoxime has been used to probe the role of myosin in cell movement (L. P. Cramer and T. J. Mitchison, J. Cell Biol. 131, 179-189 (1995)), but its specificity has been questioned (G. Steinberg and J. R. McIntosh, Eur. J. Cell Biol. 77, 284-293 (1998)). A natural product inhibitor of kinesin has been reported (R. Sakowicz et al., Science 280, 292-295 (1998)), but is thought not to be selective for different kinesins and thus is not useful for probing the role of one specific kinesin in a complex process. Hyman et al. (A. A. Hyman et al., Nature (London) 359, 533-536 (1992)) have used ATP analogs to distinguish between microtubule motility at kinetochores driven by a kinesin and a dynein, but again, this approach is unlikely to distinguish between different kinesins. Thus currently there is a lack of small molecule activators or inhibitors that are specific for one member of the kinesin family.

Therefore, there remains a need for inhibitory molecules with specificity for a particular member of a kinesin class. Such compounds would be useful as an anti-mitotic and also as an anti-cancer, anti-tumor compound. For example, as discussed above, one important member of the Kinesin Family is Eg5, a plus-end-directed motor, which is thought to generate force to push the two poles of the mitotic soindle apart. Blocking of Eg5 protein function by means of small molecules or antibodies results in the collapse of the spindle, ultimately preventing cell division. Accordingly, inhibition of mitotic kinesines, such as Eg5, is an attractive target for the development of novel anti-mitotic drugs.

### SUMMARY OF THE INVENTION

The present invention provides compounds, compositions, methods and systems for inhibiting cell growth. More specifically, the present invention provides methods, compounds and compositions that are capable of inhibiting mitosis in metabolically active cells. Compounds, and compositions of the present invention inhibit the activity of a protein involved in the assembly and maintenance of the mitotic spindle. One class of proteins which acts on the mitotic spindle is the family of mitotic kinesins, a subset of the kinesin superfamily.

In one aspect, the present invention provides a compound having the formula (I):

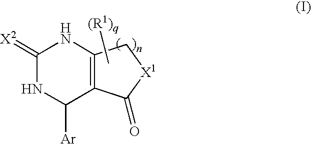

and pharmaceutically acceptable derivatives thereof; as described generally and in subclasses herein, which compounds are useful as kinesin inhibitors (e.g., Eg5 inhibitors), and thus are useful, for example, for the treatment of cancer.

In certain other embodiments, the invention provides pharmaceutical compositions comprising an inventive compound, wherein the compound is present in an amount effective to inhibit Eg5 activity. In certain other embodiments, the invention provides pharmaceutical compositions comprising an inventive compound and optionally further comprising an additional therapeutic and/or palliative agent. In yet other embodiments, the additional therapeutic agent is an anticancer agent.

In yet another aspect, the present invention provides methods for inhibiting Eg5 activity in a patient or a biological sample, comprising administering to said patient, or contacting said biological sample with an effective inhibitory amount of a compound of the invention. In still another aspect, the present invention provides methods for treating any disorder involving Eg5 activity, comprising administering to a subject in need thereof a therapeutically effective amount of a compound of the invention. In yet other embodiments, the present invention provides a method for treating cancer comprising administering to a subject in need thereof a therapeutically effective amount of a compound of the invention.

### BRIEF DESCRIPTION OF DRAWINGS

FIG. 1 depicts results of semi-empirical quantum mechanic calculation (PM3) on conformations of Monastrol.

FIG. 2 depicts data from an experiment examining the inhibition of Eg5 ATPase activity by inventive compounds.

### DEFINITIONS

Certain compounds of the present invention, and definitions of specific functional groups are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference. Furthermore, it will be appreciated by one of ordinary skill in the art that the synthetic methods, as described herein, utilize a variety of protecting groups. By the term “protecting group”, has used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group may form an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. For example, in certain embodiments, as detailed herein, certain exemplary oxygen protecting groups are utilized. These oxygen protecting groups include, but are not limited to methyl ethers, substituted methyl ethers (e.g., MOM (methoxymethyl ether), MTM (methylthiomethyl ether), BOM (benzyloxymethyl ether), PMBM (p-methoxybenzyloxymethyl ether), to name a few), substituted ethyl ethers, substituted benzyl ethers, silyl ethers (e.g., TMS (trimethylsilyl ether), TES (tiethylsilylether), TIPS (triisopropylsilyl ether), TBDMS (t-butyldimethylsilyl ether), tribenzyl silyl ether, TBDPS (t-butyldiphenyl silyl ether), to name a few), esters (e.g., formate, acetate, benzoate (Bz), trifluoroacetate, dichloroacetate, to name a few), carbonates, cyclic acetals and ketals. In certain other exemplary embodiments, nitrogen protecting groups are utilized. These nitrogen protecting groups include, but are not limited to, carbamates (including methyl, ethyl and substituted ethyl carbamates (e.g., Troc), to name a few) amides, cyclic imide derivatives, N-Alkyl and N-Aryl amines, imine derivatives, and enamine derivatives, to name a few. Certain other exemplary protecting groups are detailed herein, however, it will be appreciated that the present invention is not intended to be limited to these protecting groups; rather, a variety of additional equivalent protecting groups can be readily identified using the above criteria and utilized in the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment and prevention, for example of disorders, as described generally above. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic”, as used herein, includes both saturated and unsaturated, straight chain (i.e., unbranched), branched, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

In certain embodiments, the alkyl, alkenyl and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 aliphatic carbon atoms. Illustrative aliphatic groups thus include, but are not limited to, for example, methyl, ethyl, n-propyl, isopropyl, cyclopropyl, —CH₂-cyclopropyl, alkyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl-n, hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

The term “alkoxy” (or “alkyloxy”), or “thioalkyl” as used herein refers to an alkyl group, as previously defined, attached to the parent molecular moiety through an oxygen atom or through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkoxy, include but are not limited to, methoxy, ethoxy, propoxy, isopropoxy, n-butoxy, tert-butoxy, neopentoxy and n-hexoxy. Examples of thioalkyl include, but are not limited to, methylthio, ethylthio, propylthio, isopropylthio, n-butylthio, and the like.

The term “alkylamino” refers to a group having the structure —NHR′ wherein R′ is alkyl, as defined herein. The term “dialkylamino” refers to a group having the structure —N(R′)₂, wherein R′ is alkyl, as defined herein. The term “aminoalkyl” refers to a group having the structure NH₂R′—, wherein R′ is alkyl, as defined herein. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkylamino include, but are not limited to, methylamino, ethylamino, iso-propylamino and the like.

Some examples of substituents of the above-described aliphatic (and other) moieties of compounds of the invention include, but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl, heteroaryl, -(alkyl)aryl or -(alkyl)heteroaryl substituents described above and herein may be substituted or unsubstituted. Additional examples of generally applicable substituents are illustrated by the specific embodiments shown in the Examples that are described herein.

In general, the terms “aromatic moiety” and “heteroaromatic moiety”, as used herein, refer to stable mono- or polycyclic, heterocyclic, polycyclic, and polyheterocyclic unsaturated moieties having preferably 3-14 carbon atoms, each of which may be substituted or unsubstituted. It will also be appreciated that aromatic and heteroaromatic moieties, as defined herein may be attached via an alkyl or heteroalkyl moiety and thus also include -(alkyl)aromatic, -(heteroalkyl)aromatic, -(heteroalkyl)heteroaromatic, and -(heteroalkyl)heteroaromatic moieties. Thus, as used herein, the phrases “aromatic or heteroaromatic moieties” and “aromatic, heteroaromatic, (alkyl)aromatic, -(heteroalkyl)aromatic, (heteroalkyl)heteroaromatic, and (heteroalkyl)heteroaromatic” are interchangeable. Substituents include, but are not limited to, any of the previously mentioned substituents, i.e., the substituents recited for aliphatic moieties, or for other moieties as disclosed herein, resulting in the formation of a stable compound.

The term “aryl”, as used herein, does not differ significantly from the common meaning of the term in the art, and refers to a mono- or bicyclic carbocyclic ring system having one or two aromatic rings including, but not limited to, phenyl, naphthyl, tetrahydronaphthyl, indanyl, indenyl and the like.

The term “heteroaryl”, as used herein, does not differ significantly from the common meaning of the term in the art, and refers to a cyclic aromatic radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups (including bicyclic aryl groups) can be unsubstituted or substituted, wherein substitution includes replacement of one or more of the hydrogen atoms thereon independently with any one or more of the following moieties including, but not limited to: aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl, heteroaryl, -(alkyl)aryl or -(alkyl)heteroaryl substituents described above and herein may be substituted or unsubstituted. Additionally, it will be appreciated, that any two adjacent groups taken together may represent a 4, 5, 6, or 7-membered cyclic, substituted or unsubstituted aliphatic or heteroaliphatic moiety. Additional examples of generally applicable substituents are illustrated by the specific embodiments shown in the Examples that are described herein.

The term “cycloalkyl”, as used herein, refers specifically to cyclic moieties having three to seven, preferably three to ten carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic or heterocyclic moieties, may optionally be substituted with substituents including, but not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —OC₂(Rₓ); —CON(Rₓ)₂; —OC(O)Rₓ; —OCO₂Rₓ; —OCON(Rₓ)₂; —N(Rₓ)₂; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl, heteroaryl, -(alkyl)aryl or -(alkyl)heteroaryl substituents described above and herein may be substituted or unsubstituted. Additionally, it will be appreciated that any of the cycloaliphatic or cycloheteroaliphatic moieties described above and herein may comprise an aryl, heteroaryl, -(alkyl)aryl or -(alkyl)heteroaryl moiety fused thereto. Additional examples of generally applicable substituents are illustrated by the specific embodiments shown in the Examples that are described herein.

The term “heteroaliphatic”, as used herein, refers to aliphatic moieties in which one or more carbon atoms in the main chain have been substituted with a heteroatom. Thus, a heteroaliphatic group refers to an aliphatic chain which contains one or more oxygen sulfur, nitrogen, phosphorus or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)Rₓ; —S(O)₂Rₓ; —NRₓ(CO)Rₓ; —N(Rₓ)CO₂Rₓ; —N(Rₓ)S(O)₂Rₓ; —N(ROC(═O)N(Rₓ)₂; —S(O)₂N(Rₓ)₂; wherein each occurrence of % independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl, heteroaryl, -(alkyl)aryl or -(alkyl)heteroaryl substituents described above and herein may be substituted or unsubstituted.

In general, the term “cycloaliphatic”, as used herein, refer to a cyclic aliphatic moiety, wherein the term aliphatic is as defined above. A cycloaliphatic moiety may be substituted or unsubstituted and saturated or unsaturated. Substituents include, but are not limited to, any of the previously mentioned substituents, i.e., the substituents recited for aliphatic moieties, or for other moieties as disclosed herein, resulting in the formation of a stable compound. In certain embodiments, cycloaliphatic compounds include but are not limited to monocyclic, or polycyclic aliphatic hydrocarbons and bridged cycloalkyl compounds, which are optionally substituted with one or more functional groups. As will be appreciated by one of ordinary skill in the art, “cycloaliphatic” is intended herein to include, but is not limited to, cycloalkyl, cycloalkenyl, and cycloalkynyl moieties, which are optionally substituted with one or more functional groups. Illustrative cycloaliphatic groups thus include, but are not limited to, for example, cyclopropyl, —CH₂-cyclopropyl, cyclobutyl, —CH₂-cyclobutyl, cyclopentyl, —CH₂-cyclopentyl, cyclohexyl, —CH₂-cyclohexyl, cyclohexenylethyl, cyclohexanylethyl, norborbyl moieties and the like, which again, may bear one or more substituents.

The terms “halo” and “halogen” as used herein refer to an atom selected from fluorine, chlorine, bromine and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “acyl”, as used herein, refers to a group having the general formula —C(═O)R, where R is an aliphatic, heteroaliphatic, heterocycle, aromatic or heteroaromatic moiety, as defined herein.

The term “heterocycloalkyl”, “heterocycle” or “heterocyclic”, as used herein, refers to compounds which combine the properties of heteroaliphatic and cyclic compounds and include, but are not limited to, saturated and unsaturated mono- or polycyclic cyclic ring systems having 5-16 atoms wherein at least one ring atom is a heteroatom selected from O, S and N (wherein the nitrogen and sulfur heteroatoms may be optionally be oxidized), wherein the ring systems are optionally substituted with one or more functional groups, as defined herein. In certain embodiments, the term “heterocycloalkyl”, “heterocycle” or “heterocyclic” refers to a non-aromatic 5-, 6- or 7-membered ring or a polycyclic group wherein at least one ring atom is a heteroatom selected from O, S and N (wherein the nitrogen and sulfur heteroatoms may be optionally be oxidized), including, but not limited to, a bi- or tri-cyclic group, comprising fused six-membered rings having between one and three heteroatoms independently selected from oxygen, sulfur and nitrogen, wherein (i) each 5-membered ring has 0 to 2 double bonds, each 6-membered ring has 0 to 2 double bonds and each 7-membered ring has 0 to 3 double bonds, (ii) the nitrogen and sulfur heteroatoms may be optionally be oxidized, (iii) the nitrogen heteroatom may optionally be quaternized, and (iv) any of the above heterocyclic rings may be fused to an aryl or heteroaryl ring. Representative heterocycles include, but are not limited to, heterocycles such as furanyl, thiofuranyl, pyranyl, pyrrolyl, thienyl, pyrrolidinyl, pyrazolinyl, pyrazolidinyl, imidazolinyl, imidazolidinyl, piperidinyl, piperazinyl, oxazolyl, oxazolidinyl, isooxazolyl, isoxazolidinyl, dioxazolyl, thiadiazolyl, oxadiazolyl, tetrazolyl, triazolyl, thiatriazolyl, oxatriazolyl, thiadiazolyl, oxadiazolyl, morpholinyl, thiazolyl, thiazolidinyl, isothiazolyl, isothiazolidinyl, dithiazolyl, dithiazolidinyl, tetrahydrofuryl, and benzofused derivatives thereof. In certain embodiments, a “substituted heterocycle, or heterocycloalkyl or heterocyclic” group is utilized and as used herein, refers to a heterocycle, or heterocycloalkyl or heterocyclic group, as defined above, substituted by the independent replacement of one, two or three of the hydrogen atoms thereon with but are not limited to aliphatic; cycloaliphatic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —OC₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ; —N(Rₓ)CO₂Rₓ; —N(R_{X})S(O)₂Rₓ; —N(Rₓ)C(═O)N(Rₓ)₂; —S(O)₂N(Rₓ)₂; wherein each occurrence of Rₓ independently includes, but is not limited to, aliphatic, cycloaliphatic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, cycloaliphatic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substitutents described above and herein may be substituted or unsubstituted. Additional examples or generally applicable substituents are illustrated by the specific embodiments shown in the Examples, which are described herein.

Additionally, it will be appreciated that any of the cycloaliphatic or cycloheteroaliphatic moieties described above and herein may comprise an aryl, heteroaryl, -(alkyl)aryl or -(alkyl)heteroaryl moiety fused thereto. Additional examples of generally applicable substituents are illustrated by the specific embodiments shown in the Examples that are described herein.

As used herein, the terms “aliphatic”, “heteroaliphatic”, “alkyl”, “alkenyl”, “alkynyl”, “heteroalkyl”, “heteroalkenyl”, “heteroalkynyl”, and the like encompass substituted and unsubstituted, saturated and unsaturated, and linear and branched groups. Similarly, the terms “alicyclic”, “heterocyclic”, “heterocycloalkyl”, “heterocycle” and the like encompass substituted and unsubstituted, and saturated and unsaturated groups. Additionally, the terms “cycloalkyl”, “cycloalkenyl”, “cycloalkynyl”, “heterocycloalkyl”, “heterocycloalkenyl”, “heterocycloalkynyl”, “aromatic”, “heteroaromatic”, “aryl”, “heteroaryl” and the like encompass both substituted and unsubstituted groups.

As used herein, the term “isolated”, when applied to the compounds of the present invention, refers to such compounds that are (i) separated from at least some components with which they are associated in nature or when they are made and/or (ii) produced, prepared or manufactured by the hand of man.

The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety that is susceptible to removal in vivo yielding the parent molecule as the pharmacologically active species. An example of a pro-drug is an ester which is cleaved in vivo to yield a compound of interest. Pro-drugs of a variety of compounds, and materials and methods for derivatizing the parent compounds to create the pro-drugs, are known and may be adapted to the present invention. Certain exemplary pharmaceutical compositions and pharmaceutically acceptable derivatives will be discussed in more detail herein below.

The term “treating”, as used herein generally means that the compounds of the invention can be used in humans or animals with at least a tentative diagnosis of disease. The compounds of the invention will delay or slow the progression of the disease thereby giving the individual a more useful life span.

The term “preventing” or “prevention” as used herein means that the compounds of the present invention are useful when administered to a patient who has not been diagnosed as possibly having the disease at the time of administration, but who would normally be expected to develop the disease or be at increased risk for the disease. The compounds of the invention will slow the development of disease symptoms, delay the onset of disease, or prevent the individual from developing the disease at all.

As used herein the term “biological sample” includes, without limitation, cell cultures or extracts thereof; biopsied material obtained from an animal (e.g., mammal) or extracts thereof; and blood, saliva, urine, feces, semen, tears, or other body fluids or extracts thereof. For example, the term “biological sample” refers to any solid or fluid sample obtained from, excreted by or secreted by any living organism, including single-celled micro-organisms (such as bacteria and yeasts) and multicellular organisms (such as plants and animals, for instance a vertebrate or a mammal, and in particular a healthy or apparently healthy human subject or a human patient affected by a condition or disease to be diagnosed or investigated). The biological sample can be in any form, including a solid material such as a tissue, cells, a cell pellet, a cell extract, cell homogenates, or cell fractions; or a biopsy, or a biological fluid. The biological fluid may be obtained from any site (e.g. blood, saliva (or a mouth wash containing buccal cells), tears, plasma, serum, urine, bile, cerebrospinal fluid, amniotic fluid, peritoneal fluid, and pleural fluid, or cells therefrom, aqueous or vitreous humor, or any bodily secretion), a transudate, an exudate (e.g. fluid obtained from an abscess or any other site of infection or inflammation), or fluid obtained from a joint (e.g. a normal joint or a joint affected by disease such as rheumatoid arthritis, osteoarthritis, gout or septic arthritis). The biological sample can be obtained from any organ or tissue (including a biopsy or autopsy specimen) or may comprise cells (whether primary cells or cultured cells) or medium conditioned by any cell, tissue or organ. Biological samples may also include sections of tissues such as frozen sections taken for histological purposes. Biological samples also include mixtures of biological molecules including proteins, lipids, carbohydrates and nucleic acids generated by partial or complete fractionation of cell or tissue homogenates. Although the sample is preferably taken from a human subject, biological samples may be from any animal, plant, bacteria, virus, yeast, etc. The term animal, as used herein, refers to humans as well as non-human animals, at any stage of development, including, for example, mammals, birds, reptiles, amphibians, fish, worms and single cells. Cell cultures and live tissue samples are considered to be pluralities of animals. In certain exemplary embodiments, the non-human animal is a mammal (e.g., a rodent, a mouse, a rat, a rabbit, a monkey, a dog, a cat, a sheep, cattle, a primate, or a pig). An animal may be a transgenic animal or a human clone. If desired, the biological sample may be subjected to preliminary processing, including preliminary separation techniques.

As used herein, the phrase “radiation therapy” includes, but is not limited to, x-rays or gamma rays which are delivered from either an externally applied source such as a beam or by implantation of small radioactive sources.

As used herein, the phrase “antineoplastic agent” refers to compounds which prevent cancer cells from multiplying. In general, the antineoplastic agents of this invention prevent cancer cells from multiplying by: (1) interfering with the cell's ability to replicate DNA, or (2) inducing apoptosis in the cancerous cells.

As used herein, the term “subject” encompasses all animal species. In certain embodiments, the subject is a mammal. In certain other embodiments, the subject is a human.

“Therapeutically effective”: As used herein, the term “therapeutically effective” is defined as an amount of a compound or composition comprising the compound which is administered to an individual in need thereof to slow or cease uncontrolled or abnormal growth of cells in the individual without toxicity.

“Cancer or cancerous growth”: As used herein, the term “cancer” or “cancerous growth” means the uncontrolled, abnormal growth of cells and includes within its scope all the well known diseases that are caused by the uncontrolled and abnormal growth of cells. Non-limiting examples of common cancers include bladder cancer, breast cancer, colon cancer, endometrial cancer, head and neck cancer, lung cancer, melanoma, non-hodgkin's lymphoma, prostate cancer, and rectal cancer. A complete list of cancers is available from the National Cancer Institute (Bethesda, Md.).

### DESCRIPTION OF CERTAIN PREFERRED EMBODIMENTS OF THE INVENTION

As noted above, there remains a need for small molecule activators or inhibitors that are specific for one member of the kinesin family. Such compounds, having specificity for a particular member of a kinesin class, would be useful as an anti-mitotic and also as an anti-cancer, anti-tumorigenic compound. In certain embodiments, the present invention provides compounds, compositions, methods and systems for inhibiting cell growth. In certain exemplary embodiments, the present invention provides for methods, compounds and compositions which are capable of inhibiting mitosis in metabolically active cells. In certain embodiments, compounds, compositions and methods of the present invention inhibit the activity of a protein involved in the assembly and maintenance of the mitotic spindle. One class of proteins which acts on the mitotic spindle is the family of mitotic kinesins, a subset of the kinesin superfamily. In certain exemplary embodiments, the invention provides a new class of bicyclic dihydropyrimidines exhibiting Eg5 inhibitory activity. Thus, in certain exemplary embodiments, the present invention provides novel bicyclic dihydropyrimidine compounds useful for the treatment of cancer.

Additionally, the present invention provides pharmaceutically acceptable derivatives of the inventive compounds, and methods of treating a subject using these compounds, pharmaceutical compositions thereof, or either of these in combination with one or more additional therapeutic agents.

Monastrol

Mitchison and coworkers have demonstrated that the dihydropyrimidine-based compound monastrol is capable of arresting mammalian cells in mitosis with monopolar spindles (Mayer et al. Science 286:971-974, 1999; incorporated herein by reference). In vitro, monastrol specifically inhibited the motility of the mitotic kinesin Eg5, a motor protein required for spindle bipolarity. Monastrol was identified as causing monoastral spindles in mitotic cells in a multistep screen. See U.S. patent application Ser. No. 09/791,339 filed Feb. 23, 2001, which is hereby incorporated by reference in its entirety.

In addition, by studying the effects of monastrol and a related compound DHP2 on microtubule motility, Mayer et al. (Science 286:971-974, 1999) determined that the inhibition of monastrol on the Eg5 kinesin is specific to monastrol. Furthermore, monastrol's inhibiting effect on motility is specific for the Eg5 kinesin. Monastrol did not inhibit microtubule motility driven by conventional kinesin (Mayer et al. Science 286:971-974, 1999.)

In certain embodiments, the present invention provides constrained analogs of Monastrol having Eg5 inhibitory activity.

1) General Description of Compounds of the Invention

In certain embodiments, the compounds of the invention include compounds of the general formula (I) as further defined below:

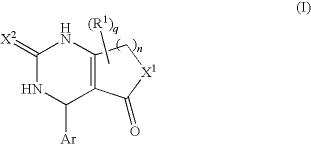

and pharmaceutically acceptable derivatives thereof;

wherein Ar is an aromatic or heteroaromatic moiety;

X¹ is O or NR^{X1}, wherein R^{X1} is hydrogen or an aliphatic, heteroaliphatic, aromatic or heteroaromatic moiety;

X² is O or S;

n is 1 or 2;

q is an integer from 0-4; and

each occurrence of R₁ is independently hydrogen, halogen, hydroxy, or an aliphatic, heteroaliphatic, aromatic or heteroaromatic moiety.

In certain embodiments, the present invention defines particular classes of compounds which are of special interest. For example, one class of compounds of special interest includes those compounds of formula (I) wherein, X¹ is O and the compound has the structure (I^{A}):

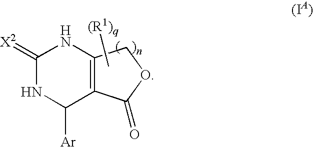

Another class of compounds of special interest includes those compounds of formula (I) wherein, X¹ is NR^{X1} and the compound has the Formula (I^{B}):

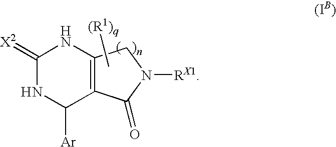

Another class of compounds of special interest includes those compounds of formula (I) wherein, X² is O and the compound has the Formula (I^{c}):

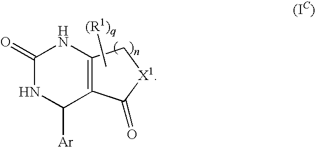

Another class of compounds: of special interest includes those compounds of formula (I) wherein, X² is S and the compound has the Formula (I^{D}):

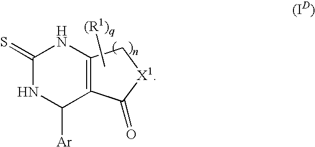

Another class of compounds of special interest includes those compounds of formula (I) wherein, Ar is a phenyl or pyridyl moiety and the compound has the Formula (I^{E}):

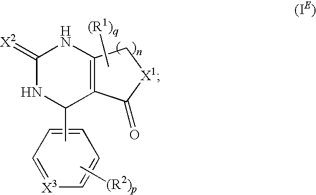

wherein X³ is N or CR²; p is 0-5; and each occurrence of R² is independently hydrogen, halogen, CN, NO₂, alkyl, alkenyl, alkynyl, heteroalkyl, heteroalkenyl, heteroalkynyl, cycloalkyl, heterocycle, aryl, heteroaryl, -(alkyl)cycloalkyl, -(alkyl)heterocycle, -(heteroalkyl)cycloalkyl, -(heteroalkyl)heterocycle, -(alkyl)aryl, -(heteroalkyl)aryl, -(alkyl)heteroaryl, -(heteroalkyl)heteroaryl, —OR^{2A}, —S(═O)ₓR^{2A}, —N(R^{2A})₂, —SO₂N(R^{2A})₂, —C(═O)R^{2A}, —C(═O)N(R^{2A})₂, C(═O)OR^{2A}, —N(R^{2A})C(═O)R^{2B} or —N(R^{2A})SO₂R^{2B}, wherein x is 0, 1 or 2; and each occurrence of R^{2A} and R^{2B} is independently hydrogen, lower alkyl, lower heteroalkyl, aryl, heteroaryl, -(alkyl)aryl or -(alkyl)heteroaryl; or R^{2A} and R^{2B}, taken together with the atoms to which they are attached, form a substituted or unsubstituted 5-8 membered heterocyclic moiety.

In certain embodiments, the following groups do not occur simultaneously as defined: X¹ and X² are each 0, n is 1 and q is 0.

In certain embodiments, the following groups do not occur simultaneously as defined: X² is O, n is 1, q is 0 and X¹ is NR^{X1}, wherein R^{X1} is H, methyl of —CH₂Ph.

A number of important subclasses of each of the foregoing classes deserve separate mention; these subclasses include subclasses of the foregoing classes in which:

i) X¹ is O;

ii) X¹ is NR^{X1}, wherein R^{X1} is hydrogen, alkyl, alkenyl, alkynyl, heteroalkyl, heteroalkenyl, heteroalkynyl, cycloalkyl, heterocycle, acyl, aryl, heteroaryl, -(alkyl)cycloalkyl, -(alkyl)heterocycle, -(heteroalkyl)cycloalkyl, (heteroalkyl)heterocycle, -(alkyl)aryl, -(heteroalkyl)aryl, -(alkyl)heteroaryl or -(heteroalkyl)heteroaryl;

iii) X¹ is NR^{X1}, wherein R^{X1} is hydrogen, lower alkyl, lower heteroalkyl, cycloalkyl, heterocycle, lower acyl, aryl, heteroaryl, -(alkyl)cycloalkyl, -(alkyl)heterocycle, -(alkyl)aryl, -(heteroalkyl)aryl or -(alkyl)heteroaryl;

iv) X¹ is NR^{X1}, wherein R^{X1} is hydrogen, lower alkyl, lower acyl, aryl or heteroaryl;

v) X¹ is NR^{X1}, wherein R^{X1} is hydrogen, lower alkyl or lower acyl;

vi) X¹ is NH;

vii) X¹ is NAc;

viii) X¹ is NR^{X1}, wherein R^{X1} is methyl, ethyl, propyl or iso-propyl;

ix) X² is O;

x) X² is S;

xi) n is 1;

xii) n is 2;

xiii) p is 0;

xiv) p is 1;

xv) p is 2;

xvi) p is 3;

xvii) q is 0;

xviii) q is 1;

xix) q is 2;

xx) q is 3;

xxi) q is 4;

xxii) n is 1 and q is 0;

xxiii) n is 2 and q is 0;

xxiv) each occurrence of R¹ is independently hydrogen, halogen, hydroxy, alkyl, alkenyl, alkynyl, heteroalkyl, heteroalkenyl, heteroalkynyl, cycloalkyl, heterocycle, aryl, heteroaryl, -(alkyl)cycloalkyl, -(alkyl)heterocycle, -(heteroalkyl)cycloalkyl, -(heteroalkyl)heterocycle, -(alkyl)aryl, -(heteroalkyl)aryl, -(alkyl)heteroaryl, -(heteroalkyl)heteroaryl, —OR^{1A}, —SR^{1A}, —N(R^{1A})₂, —SO₂NR^{1A})₂, —C(═O)N(R^{1A})₂, halogen, —CN, —NO₂, —C(═O)R^{1A}, —C(═O)OR^{1A}, —N(R^{1A})C(═O)R^{1B} or —N(R^{1A})SO₂R^{1B}, wherein each occurrence of R^{1A} and R^{1B} is independently hydrogen, lower alkyl, lower heteroalkyl, aryl, heteroaryl, -(alkyl)aryl or -(alkyl)heteroaryl; or R^{1A} and R^{1B}, taken together with the atoms to which they are attached, form a substituted or unsubstituted 5-8 membered heterocyclic moiety;

xxv) each occurrence of R¹ is independently hydrogen, halogen, hydroxy, lower alkyl, lower heteroalkyl, 3-6 membered cycloalkyl, 3-6 membered heterocycle, aryl, heteroaryl, -(lower alkyl)cycloalkyl, -(lower alkyl)heterocycle, -(lower alkyl)aryl or -(lower alkyl)heteroaryl;

xxvi) each occurrence of R¹ is independently lower alkyl;

xxvii) each occurrence of R¹ is independently methyl, ethyl, propyl, i-propyl, butyl or t-butyl;

xxviii) q is 1 and R¹ is methyl;

xxix) q is 2 and each occurrence of R¹ is lower alkyl;

xxx) q is 2 and each occurrence of R¹ is methyl;

xxxi) R¹, for each occurrence, is hydrogen;

xxxii) each occurrence of R² is independently hydrogen, halogen, CN, NO₂, alkyl, alkenyl, alkynyl, heteroalkyl, heteroalkenyl, heteroalkynyl, cycloalkyl, heterocycle, aryl, heteroaryl, -(alkyl)cycloalkyl, -(alkyl)heterocycle, -(heteroalkyl)cycloalkyl, -(heteroalkyl)heterocycle, -(alkyl)aryl, -(heteroalkyl)aryl, -(alkyl)heteroaryl, -(heteroalkyl)heteroaryl, —OR^{2A}, —S(═O)ₓR^{2A}, —N(R^{2A})₂, —SO₂N(R^{2A})₂, —C(═O)R^{2A}, —C(═O)N(R^{2A})₂, —C(═O)OR^{2A}, —N(R^{2A})C(═O))R^{2B} or —N(R^{2A})SO₂R^{2B}, wherein x is 0, 1 or 2; and each occurrence of R^{2A} and R^{2B} is independently hydrogen, lower alkyl, lower heteroalkyl, aryl, heteroaryl, -(alkyl)aryl or -(alkyl)heteroaryl; or R^{2A} and R^{2B}, taken together with the atoms to which they are attached, form a substituted or unsubstituted 5-8 membered heterocyclic moiety;

xxxiii) each occurrence of R² is independently hydrogen, halogen, CN, NO₂, lower alkyl, lower heteroalkyl, 3-6 membered cycloalkyl, 3-8 membered heterocycle, aryl, heteroaryl, -(lower alkyl)cycloalkyl, -(lower alkyl)heterocycle, -(lower heteroalkyl)cycloalkyl, -(lower heteroalkyl)heterocycle, -(lower alkyl)aryl, -(lower heteroalkyl)aryl, -(lower alkyl)heteroaryl , -(lower heteroalkyl)heteroaryl, —OR^{2A}, —SR^{2A}, —N(R^{2A})₂, —C(═O)R^{2A}, —C(═O)N(R^{2A})₂, —C(═O)OR^{2A}, —N(R^{2A})C(═O)R^{2B} or —N(R^{2A})SO₂R^{2B}, wherein each occurrence of R^{2A} and R^{2B} is independently hydrogen, lower alkyl, lower heteroalkyl, aryl, heteroaryl, -(lower alkyl)aryl or -(lower alkyl)heteroaryl; or R^{2A} and R^{2B}, taken together with the atoms to which they are attached, form a substituted or unsubstituted 5-8 membered heterocyclic moiety;

xxxiv) each occurrence of R² is independently hydrogen, halogen, lower alkyl, lower heteroalkyl, —OR^{2A}, —SR^{2A}, —N(R^{2A})₂, —C(═O)R^{2A}, —C(═O)N(R^{2A})₂, —C(═O)OR^{2A}, —N(R^{2A})C(═O)R^{2B} or —N(R^{2A})SO₂R^{2B}, wherein each occurrence of R^{2A} and R^{2B} is independently hydrogen, lower alkyl, lower heteroalkyl, aryl, heteroaryl, -(lower alkyl)aryl or -(lower alkyl)heteroaryl; or R^{2A} and R^{2B}, taken together with the atoms to which they are attached, form a substituted or unsubstituted 5-8 membered heterocyclic moiety;

xxxv) each occurrence of R² is independently hydrogen, halogen, lower alkyl or —OR^{2A}; wherein R^{2A} is hydrogen, lower alkyl, aryl, heteroaryl, -(lower alkyl)aryl or -(lower alkyl)heteroaryl;

xxxvi) each occurrence of R² is independently hydrogen, halogen, lower alkyl or —OR^{2A}; wherein R^{2A} is hydrogen, or lower alkyl;

xxxvii) each occurrence of R² is independently hydrogen, hydroxyl or lower alkoxy;

xxxviii) p is 1 and R² is hydroxyl or lower alkoxy;

xxxix) p is 1 and R² is meta-hydroxyl or meta-lower alkoxy;

xl) p is 1 and R² is meta-hydroxyl;

xli) Ar is substituted or unsubstituted phenyl or naphthyl, or a moiety having one of the following structures:

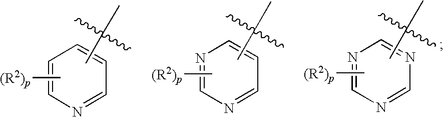

wherein p is 0-5 and R² takes the definition given in any one of subsets xxxii)-xl);

xlii) Ar is substituted or unsubstituted phenyl or pyridyl;

xliii) Ar has one of the following structures:

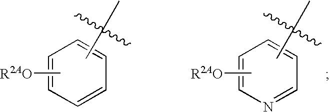

wherein R^{2A} is hydrogen, alkyl, alkenyl, alkynyl, heteroalkyl, heteroalkenyl, heteroalkynyl, cycloalkyl, heterocycle, acyl, aryl, heteroaryl, -(alkyl)cycloalkyl, -(alkyl)heterocycle, -(heteroalkyl)cycloalkyl, -(heteroalkyl)heterocycle, -(alkyl)aryl, -(heteroalkyl)aryl, -(alkyl)heteroaryl, -(heteroalkyl)heteroaryl or an oxygen protecting group;

xliv) Ar has one of the following structures:

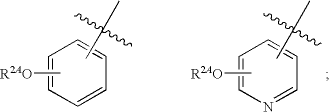

wherein R^{2A} is hydrogen, lower alkyl, lower heteroalkyl, cycloalkyl, heterocycle, lower acyl, aryl, heteroaryl, -(alkyl)cycloalkyl, -(alkyl)heterocycle, -(alkyl)aryl, -(heteroalkyl)aryl, -(alkyl)heteroaryl or an oxygen protecting group;

xlv) Ar has one of the following structures:

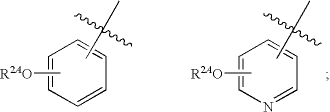

wherein R^{2A} is hydrogen, lower alkyl, lower acyl, aryl, heteroaryl or an oxygen protecting group;

xlvi) Ar has one of the following structures:

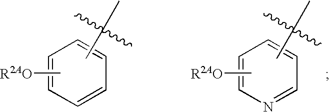

wherein R^{2A} is hydrogen, lower alkyl, lower acyl or an oxygen protecting group;

xlvii) Ar has one of the following structures:

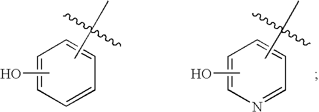

xlviii) Ar has the structure:

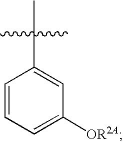

wherein R^{2A} is hydrogen, alkyl, alkenyl, alkynyl, heteroalkyl, heteroalkenyl, heteroalkynyl, cycloalkyl, heterocycle, acyl, aryl, heteroaryl, -(alkyl)cycloalkyl, -(alkyl)heterocycle, -(heteroalkyl)cycloalkyl, -(heteroalkyl)heterocycle, -(alkyl)aryl, -(heteroalkyl)aryl, -(alkyl)heteroaryl , -(heteroalkyl)heteroaryl or an oxygen protecting group;

xlix) Ar has the structure:

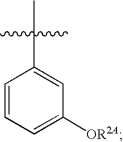

wherein R^{2A} is hydrogen, lower alkyl, lower heteroalkyl, cycloalkyl, heterocycle, lower acyl, aryl, heteroaryl, -(alkyl)cycloalkyl, -(alkyl)heterocycle, -(alkyl)aryl, -(heteroalkyl)aryl, -(alkyl)heteroaryl or an oxygen protecting group;

1) Ar has the structure:

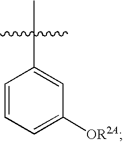

wherein R^{2A} is hydrogen, lower alkyl, lower acyl, aryl, heteroaryl or an oxygen protecting group;

li) Ar has the structure:

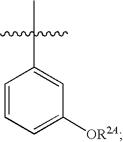

wherein R^{2A} is hydrogen, lower alkyl, lower acyl or an oxygen protecting group; and/or

lii) Ar has the structure:

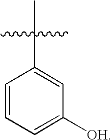

It will be appreciated that for each of the classes and subclasses described above and herein, any one or more occurrences of aliphatic or heteroaliphatic may independently be substituted or unsubstituted, cyclic or acyclic, linear or branched, saturated or unsaturated and any one or more occurrences of aryl, heteroaryl, cycloaliphatic, cycloheteroaliphatic may be substituted or unsubstituted.

The reader will also appreciate that all possible combinations of the variables described in i)-through lii) above (e.g., R¹, R², X¹, X², n, p and q, among others) are considered part of the invention. Thus, the invention encompasses any and all compounds of formula I generated by taking any possible permutation of variables R¹, R², X¹, X², n, p and q, and other variables/substituents (e.g., R^{X1}, R^{1A}, R^{1BA}, R^{2A}, etc.) as further defined for R¹, R², X¹, described in i)-through lii) above.

For example, an exemplary combination of variables described in i)-through lii) above includes those compounds of Formula I wherein:

X¹ is O;

X² is S;

each occurrence of R¹ is independently hydrogen, alkyl, alkenyl, alkynyl, heteroalkyl, heteroalkenyl, heteroalkynyl, cycloalkyl, heterocycle, aryl, heteroaryl, -(allypcycloalkyl, -(alkyl)heterocycle, -(heteroalkyl)cycloalkyl, (heteroalkyl)heterocycle, -(alkyl)aryl, -(heteroalkyl)aryl, -(alkyl)heteroaryl , -(heteroalkyl)heteroaryl, —OR^{1A}, —S(═O)ₓR^{1A}, —N(R^{1A})₂, —SO₂N(R^{1A})₂, —C(═O)R^{1A}, —C(═O)N(R^{1A})₂, halogen, —CN, —NO₂, —C(═O)OR^{1A}, —N(R^{1A})C(═O)R^{1B} or —N(R^{1A})SO₂R^{1B}, wherein x is 0, 1 or 2; and each occurrence of R^{1A} and R^{1B} is independently hydrogen, lower alkyl, lower heteroalkyl, aryl, heteroaryl, -(alkyl)aryl or -(alkyl)heteroaryl; or R^{1A} and R^{1B}, taken together with the atoms to which they are attached, form a substituted or unsubstituted 5-8 membered heterocyclic moiety;

n is 1 or 2;

q is 0;

Ar is substituted or unsubstituted phenyl or naphthyl, or a moiety having one of the following structures:

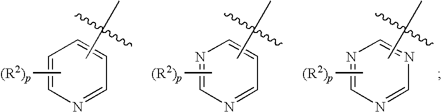

wherein p is 1; and each occurrence of R² is independently hydrogen, halogen, CN, NO₂, alkyl, alkenyl, alkynyl, heteroalkyl, heteroalkenyl, heteroalkynyl, cycloalkyl, heterocycle, aryl, heteroaryl, -(alkyl)cycloalkyl, -(alkyl)heterocycle, -(heteroalkyl)cycloalkyl, -(heteroalkyl)heterocycle, -(alkyl)aryl, -(heteroalkyl)aryl, -(alkyl)heteroaryl, -(heteroalkyl)heteroaryl, —OR^{2A}, —S(═O)ₓR^{2A}, —N(R^{2A})₂, —SO₂N(R^{2A})₂, —C(═O)R^{1A}, —C(═O)N(R^{2A})₂, —C(═O)OR^{2A}, —N(R^{2A})C(═O)R^{2B} or N(R^{2A})SO₂R^{2B}, wherein x is 0, 1 or 2; and each occurrence of R^{2A} and R^{2B} is independently hydrogen, lower alkyl, lower heteroalkyl, aryl, heteroaryl, -(alkyl)aryl or -(alkyl)heteroaryl; or R^{2A} and R^{2B}, taken together with the atoms to which they are attached, form a substituted or unsubstituted 5-8 membered heterocyclic moiety.

Other exemplary combinations are illustrated by compounds of the following subgroups I-XII:

I. Compounds having the structure (and pharmaceutically acceptable derivatives thereof):

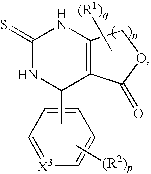

wherein n, p, q, R¹, R², and X³ are as defined generally and in classes and subclasses herein.

II. Compounds having the structure (and pharmaceutically acceptable derivatives thereof):

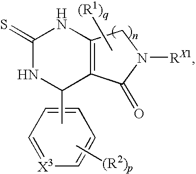

wherein n, p, q, R¹, R², and X³ are as defined generally and in classes and subclasses herein; and R^{X1} is hydrogen, lower alkyl, acyl or a nitrogen protecting group.

III. Compounds Having the Structure (and Pharmaceutically Acceptable Derivatives Thereof):

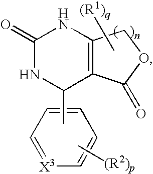

wherein n, p, q, R¹, R², and X³ are as defined generally and in classes and subclasses herein.

IV. Compounds Having the Structure (and Pharmaceutically Acceptable Derivatives Thereof):

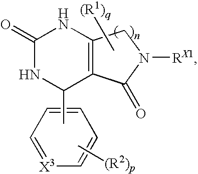

wherein n, p, q, R¹, R², and X³ are as defined generally and in classes and subclasses herein; and R^{X1} is hydrogen, lower alkyl, acyl or a nitrogen protecting group.

V. Compounds Having the Structure (and Pharmaceutically Acceptable Derivatives Thereof):

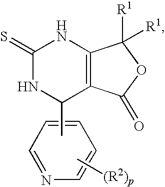

wherein p and R² are as defined generally and in classes and subclasses herein and each occurrence of R¹ is independently hydrogen, halogen, lower alkyl or lower haloalkyl. In certain embodiments, each occurrence of R¹ is hydrogen. In certain embodiments, each occurrence of R¹ is independently halogen, lower alkyl or lower haloalkyl. In certain embodiments, each occurrence of R¹ is independently lower alkyl. In certain embodiments, each occurrence of R¹ is methyl. In certain embodiments, p is 0. In certain other embodiments, p is 1. In yet other embodiments, p is 1 and R² is halogen, lower alkyl, hydroxy or lower alkoxy. In certain other embodiments, p is 1 and R² is meta-hydroxyl.

VI. Compounds Having the Structure (and Pharmaceutically Acceptable Derivatives Thereof):

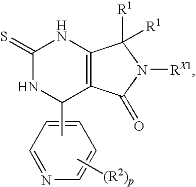

wherein p and R² are as defined generally and in classes and subclasses herein; each occurrence of R¹ is independently hydrogen, halogen, lower alkyl or lower haloalkyl and R^{X1} is hydrogen, lower alkyl, acyl or a nitrogen protecting group. In certain embodiments, each occurrence of R¹ is hydrogen. In certain embodiments, each occurrence of R¹ is independently halogen, lower alkyl or lower haloalkyl. In certain embodiments, each occurrence of R¹ is independently lower alkyl. In certain embodiments, each occurrence of R¹ is methyl. In certain embodiments, p is 0. In certain other embodiments, p is 1. In yet other embodiments, p is 1 and R² is halogen, lower alkyl, hydroxy or lower alkoxy. In certain other embodiments, p is 1 and R² is meta-hydroxyl.

VII. Compounds Having the Structure (and Pharmaceutically Acceptable Derivatives Thereof):

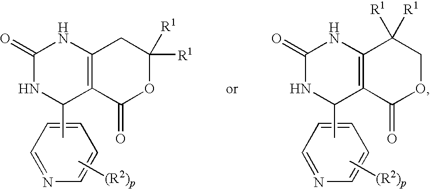

wherein p and R² are as defined generally and in classes and subclasses herein and each occurrence of R¹ is independently hydrogen, halogen, lower alkyl or lower haloalkyl. In certain embodiments, each occurrence of R¹ is hydrogen. In certain embodiments, each occurrence of R¹ is independently halogen, lower alkyl or lower haloalkyl. In certain embodiments, each occurrence of R¹ is independently lower alkyl. In, certain embodiments, each occurrence of R¹ is methyl. In certain embodiments, p is 0. In certain other embodiments, p is 1. In yet other embodiments, p is 1 and R² is halogen, lower alkyl, hydroxy or lower alkoxy. In certain other embodiments, p is 1 and R² is meta-hydroxyl. In certain exemplary embodiments, R^{X1} is hydrogen.

VIII. Compounds Having the Structure (and Pharmaceutically Acceptable Derivatives Thereof):

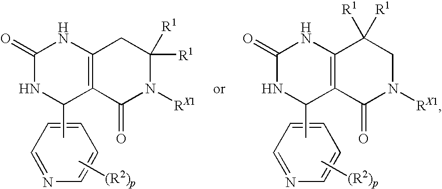

wherein p and R² are as defined generally and in classes and subclasses herein; each occurrence of R¹ is independently hydrogen, halogen, lower alkyl or lower haloalkyl and R^{X1} is hydrogen, lower alkyl, acyl or a nitrogen protecting group. In certain embodiments, each occurrence of R¹ is hydrogen. In certain embodiments, each occurrence of R¹ is independently halogen, lower alkyl or lower haloalkyl. In certain embodiments, each occurrence of R¹ is independently lower alkyl. In certain embodiments, each occurrence of R¹ is methyl. In certain embodiments, p is 0. In certain other embodiments, p is 1. In yet other embodiments, p is 1 and R² is halogen, lower alkyl, hydroxy or lower alkoxy. In certain other embodiments, p is 1 and R² is meta-hydroxyl. In certain exemplary embodiments, R^{X1} is hydrogen. In certain exemplary embodiments, R^{X1} is hydrogen.

IX. Compounds Having the Structure (and Pharmaceutically Acceptable Derivatives Thereof):

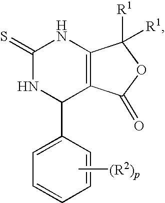

wherein p and R² are as defined generally and in classes and subclasses herein and each occurrence of R¹ is independently hydrogen, halogen, lower alkyl or lower haloalkyl. In certain embodiments, each occurrence of R¹ is hydrogen. In certain embodiments, each occurrence of R¹ is independently halogen, lower alkyl or lower haloalkyl. In certain embodiments, each occurrence of R¹ is independently lower alkyl. In certain embodiments, each occurrence of R¹ is methyl. In certain embodiments, p is 0. In certain other embodiments, p is 1. In yet other embodiments, p is 1 and R² is halogen, lower alkyl, hydroxy or lower alkoxy. In certain other embodiments, p is 1 and R² is meta-hydroxyl

X. Compounds Having the Structure (and Pharmaceutically Acceptable Derivatives Thereof):

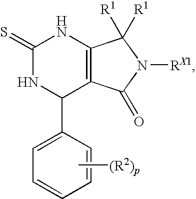

wherein p and R² are as defined generally and in classes and subclasses herein; each occurrence of R¹ is independently hydrogen, halogen, lower alkyl or lower haloalkyl and R^{X1} is hydrogen, lower alkyl, acyl or a nitrogen protecting group. In certain embodiments, each occurrence of R¹ is hydrogen. In certain embodiments, each occurrence of R¹ is independently halogen, lower alkyl or lower haloalkyl. In certain embodiments, each occurrence of R¹ is independently lower alkyl. In certain embodiments, each occurrence of R¹ is methyl. In certain embodiments, p is 0. In certain other embodiments, p is 1. In yet other embodiments, p is 1 and R² is halogen, lower alkyl, hydroxy or lower alkoxy. In certain exemplary embodiments, p is 1 and R² is meta-hydroxyl.

XI. Compounds Having the Structure (and Pharmaceutically Acceptable Derivatives Thereof):

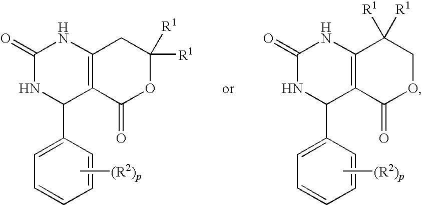

wherein p and R² are as defined generally and in classes and subclasses herein and each occurrence of R¹ is independently hydrogen, halogen, lower alkyl or lower haloalkyl. In certain embodiments, each occurrence of R¹ is hydrogen. In certain embodiments, each occurrence of R¹ is independently halogen, lower alkyl or lower haloalkyl. In certain embodiments, each occurrence of R¹ is independently lower alkyl. In certain embodiments, each occurrence of R¹ is methyl. In certain embodiments, p is 0. In certain other embodiments, p is 1. In yet other embodiments, p is 1 and R² is halogen, lower alkyl, hydroxy or lower alkoxy. In certain exemplary embodiments, p is 1 and R² is meta-hydroxyl. In certain exemplary embodiments, R^{X1} is hydrogen.

XII. Compounds Having the Structure (and Pharmaceutically Acceptable Derivatives Thereof):

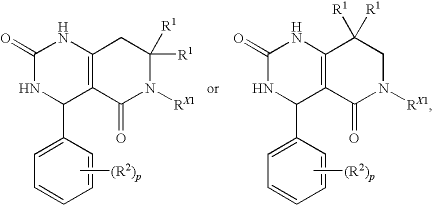

wherein p and R² are as defined generally and in classes and subclasses herein; each occurrence of R¹ is independently hydrogen, halogen, lower alkyl or lower haloalkyl and R^{X1} is hydrogen, lower alkyl, acyl or a nitrogen protecting group. In certain embodiments, each occurrence of R¹ is hydrogen. In certain embodiments, each occurrence of R¹ is independently halogen, lower alkyl or lower haloalkyl. In certain embodiments, each occurrence of R¹ is independently lower alkyl. In certain embodiments, each occurrence of R¹ is methyl. In certain embodiments, p is 0. In certain other embodiments, p is 1. In yet other embodiments, p is 1 and R² is halogen, lower alkyl, hydroxy or lower alkoxy. In certain exemplary embodiments, p is 1 and R² is meta-hydroxyl. In certain exemplary embodiments, R^{X1} is hydrogen.

It will also be appreciated that for each of the subgroups described above, a variety of other subclasses are of special interest, including, but not limited to those classes described above i)-lii) and classes, subclasses and species of compounds described above and in the examples herein.

Some of the foregoing compounds can comprise one or more asymmetric centers, and thus can exist in various isomeric forms, e.g., stereoisomers and/or diastereomers. Thus, inventive compounds and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain other embodiments, mixtures of stereoisomers or diastereomers are provided. For example, compounds having the following stereochemistry are provided:

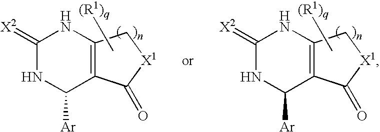

or mixture of these stereoisomers.

Furthermore, certain compounds, as described herein may have one or more double bonds that can exist as either the Z or E isomer, unless otherwise indicated. The invention additionally encompasses the compounds as individual isomers substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds of the invention and one or more pharmaceutically acceptable excipients or additives.

Compounds of the invention may be prepared by crystallization of compound of formula (I) under different conditions and may exist as one or a combination of polymorphs of compound of general formula (I) forming part of this invention. For example, different polymorphs may be identified and/or prepared using different solvents, or different mixtures of solvents for recrystallization; by performing crystallizations at different temperatures; or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other techniques. Thus, the present invention encompasses inventive compounds, their derivatives, their tautomeric forms, their stereoisomers, their polymorphs, their pharmaceutically acceptable salts their pharmaceutically acceptable solvates and pharmaceutically acceptable compositions containing them.

2) Synthetic Overview:

The practitioner has a well-established literature of heterocycle chemistry to draw upon, in combination with the information contained herein, for guidance on synthetic strategies, protecting groups, and other materials and methods useful for the synthesis of the compounds of this invention, including compounds containing the various R¹, R² and Ar substituents and X¹, X² and X³ moieties.

The various references cited herein provide helpful background information on preparing compounds similar to the inventive compounds described herein or relevant intermediates, as well as information on formulation, uses, and administration of such compounds which may be of interest.

Moreover, the practitioner is directed to the specific guidance and examples provided in this document relating to various exemplary compounds and intermediates thereof.

As described above, the present invention provides novel compounds, specifically compounds having the following general structure:

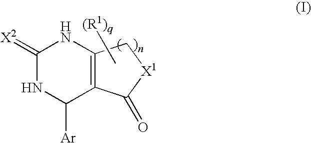

and pharmaceutically acceptable derivatives thereof;

wherein n, p, Ar, R¹, X¹ and X² are as defined generally above and in classes and subclasses herein.

A synthesis of Monastrol, previously reported by Biginelli and co-workers, is depicted in Scheme 1 (see, C. O. Kappe, Tetrahedron 1993, 49, 6937-6963).

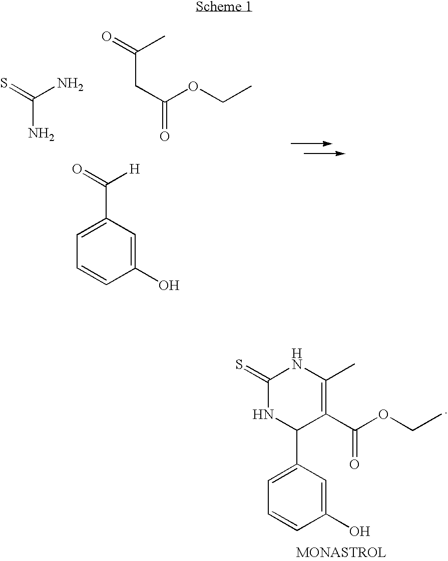

In analogy to Biginelli's three-component synthesis of Monastrol, it is proposed that compounds of the invention may be accessible via a similar approach, as depicted in Scheme 2.

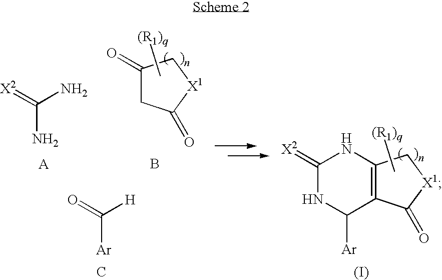

wherein Ar is an aromatic or heteroaromatic moiety;

X¹ is O or NR^{X1}, wherein R^{X1} is hydrogen or an aliphatic, heteroaliphatic, aromatic or heteroaromatic moiety;

X² is O or S;

n is 1 or 2;

q is an integer from 0-4; and

each occurrence of R₁ is independently hydrogen, or an aliphatic, heteroaliphatic, aromatic or heteroaromatic moiety.

However, published reports indicate that subjecting 5-membered esters (i.e., component B where n is 1 and X¹ is O) to Biginelli reaction conditions did not yield the desired products (See, for example, G. Byk, H. E. Gottlieb, J. Herscovici, F. Mirkin, J Comb Chem 2000, 2, 732-735).

An alternative approach comprises effecting Biginelli's reaction with an open-chain component B, as depicted in Scheme 3. Ring closure of dihydropyrimidine intermediate D may then be effected to yield compounds of general formula (I).

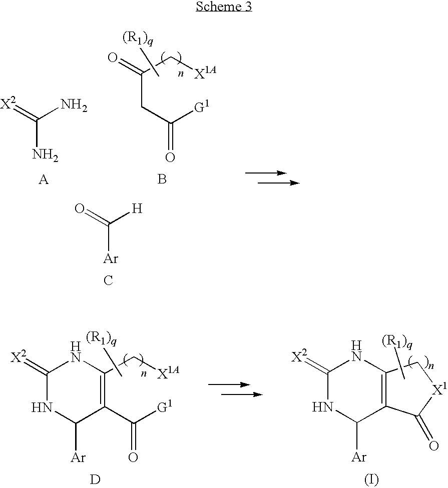

wherein n is 1 or 2; Ar is an aromatic or heteroaromatic moiety; X^{1A} is —OR^{X1B} or —N^{X1C}R^{X1}, where R^{X1B} is hydrogen or an oxygen protecting group, R^{X1C} is hydrogen or a nitrogen protecting group and R^{X1} is hydrogen, alkyl, alkenyl, alkynyl, heteroalkyl, heteroalkenyl, heteroalkynyl, cycloalkyl, heterocycle, acyl, aryl, heteroaryl, -(alkyl)cycloalkyl, -(alkyl)heterocycle, -(heteroalkyl)cycloalkyl, -(heteroalkyl)heterocycle, -(alkyl)aryl, -(heteroalkyl)aryl, -(alkyl)heteroaryl or -(heteroalkyl)heteroaryl; and G¹ is a leaving group.

In certain embodiments, R^{X1} is hydrogen, lower alkyl, lower heteroalkyl, cycloalkyl, heterocycle, lower acyl, aryl, heteroaryl, -(alkyl)cycloalkyl, -(alkyl)heterocycle, -(alkyl)aryl, -(heteroalkyl)aryl or -(alkyl)heteroaryl. In certain other embodiments, R^{X1} is hydrogen, lower alkyl, lower acyl; aryl or heteroaryl. In certain other embodiments, R^{X1} is hydrogen, lower alkyl or lower acyl. In certain exemplary embodiments, R^{X1} is hydrogen.

In certain other embodiments, G¹ is halogen or —OR^{G1A}, wherein R^{G1A} is alkyl, alkenyl, alkynyl, heteroalkyl, heteroalkenyl, heteroalkynyl, cycloalkyl, heterocycle, aryl, heteroaryl, -(alkyl)cycloalkyl, -(alkyl)heterocycle, -(heteroalkyl)cycloalkyl, -(heteroalkyl)heterocycle, -(alkyl)aryl, -(heteroalkyl)aryl, -(alkyl)heteroaryl or -(heteroalkyl)heteroaryl. In certain other embodiments, G¹ is halogen or wherein R^{G1A} is lower alkyl, lower heteroalkyl, cycloalkyl, heterocycle, aryl, heteroaryl, -(alkyl)cycloalkyl, -(alkyl)heterocycle, -(alkyl)aryl, -(heteroalkyl)aryl or -(alkyl)heteroaryl. In certain other embodiments, G¹ is halogen or —OR^{G1A}, wherein R^{G1A} is lower alkyl, aryl or heteroaryl. In certain other embodiments, G¹ is —OR^{G1A}, wherein R^{G1A} is lower alkyl.

In certain embodiments, R^{X1B} is alkyl, alkenyl, alkynyl, heteroalkyl, heteroalkenyl, heteroalkynyl, aryl, heteroaryl, silyl, —C(═O)R^{x}, —C(═S)R^{x}, —C(═NR^{x})R^{y}, —SO₂R^{x}, wherein R^{x} and R^{y} are each independently hydrogen, alkyl, alkenyl, alkynyl, cycloalkyl, cycloalkenyl, cycloalkynyl, heteroalkyl, heteroalkenyl, heteroalkynyl, heterocycloalkyl, heterocycloalkenyl, heterocycloalkynyl, heteroaliphatic, heteroalicyclic, aryl, heteroaryl, —C(═O)R^{A} or —ZR^{A}, wherein Z is —O—, —S—, —NR^{B}, wherein each occurrence of R^{A} and R^{B} is independently hydrogen, or an alkyl, alkenyl, alkynyl, cycloalkyl, cycloalkenyl, cycloalkynyl, heteroalkyl, heteroalkenyl, heteroalkynyl, heterocycloalkyl, heterocycloalkenyl, heterocycloalkynyl, heteroaliphatic, heteroalicyclic, aryl or heteroaryl moiety. In certain exemplary embodiments, R^{X1B} is a silyl protecting group. In certain exemplary embodiments, R^{X1B} is a trialkylsilyl protecting group. In certain exemplary embodiments, R^{X1B} is triisopropylsilyl.

In certain embodiments, R^{X1C} is alkyl, alkenyl, —C(═O)R^{x}, —C(═O)OR^{x}, —SR^{x}, SO₂R^{x}, or R^{X1} and R^{X1C}, taken together form a moiety having the structure ═CR^{x}R^{y}, wherein R^{X1} and R^{X1C} are not simultaneously hydrogen and R^{x} and R^{y} are each independently hydrogen, alkyl, alkenyl, alkynyl, cycloalkyl, cycloalkenyl, cycloalkynyl, heteroalkyl, heteroalkenyl, heteroalkynyl, heterocycloalkyl, heterocycloalkenyl, heterocycloalkynyl, heteroaliphatic, heteroalicyclic, aryl, heteroaryl, —C(═O)R^{A} or —ZR^{A}, wherein Z is —O—, —S—, —NR^{B}, wherein each occurrence of R^{A} and R^{B} is independently hydrogen, or an alkyl, alkenyl, alkynyl, cycloalkyl, cycloalkenyl, cycloalkynyl, heteroalkyl, heteroalkenyl, heteroalkynyl, heterocycloalkyl, heterocycloalkenyl, heterocycloalkynyl, heteroaliphatic, heteroalicyclic, aryl or heteroaryl moiety. In certain exemplary embodiments, R^{X1} is hydrogen and R^{X1C} is —C(═O)R^{x}, wherein R^{x} is substituted or unsubstituted lower alkyl or lower alkoxy. In certain exemplary embodiments, R^{X1} is hydrogen and R^{X1C} is —C(═O)R^{x}, wherein R^{x} is tert-Butoxy.

Thus, in certain embodiments, the inventive method comprises

i) reacting a compound having the structure:

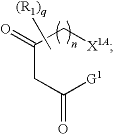

wherein n, q and R¹ are as defined generally above and in classes and subclasses herein,

X^{1A} is —OR^{X1B} or —NR^{X1C}R^{X1}, where R^{X1B} is hydrogen or an oxygen protecting group, R^{X1C} is hydrogen or a nitrogen protecting group and R^{X1} is hydrogen, alkyl, alkenyl, alkynyl, heteroalkyl, heteroalkenyl, heteroalkynyl, cycloalkyl, heterocycle, acyl, aryl, heteroaryl, -(alkyl)cycloalkyl, -(alkyl)heterocycle, -(heteroalkyl)cycloalkyl, -(heteroalkyl)heterocycle, -(alkyl)aryl, -(heteroalkyl)aryl, -(alkyl)heteroaryl or -(heteroalkyl)heteroaryl; and G¹ is a leaving group;

with a compound having the structure:

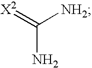

wherein X² is O or S;

and a compound having the structure:

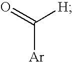

wherein Ar is an aromatic or heteroaromatic moiety; under suitable conditions to give an intermediate having the structure:

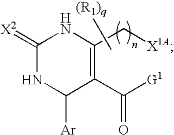

ii) reacting the intermediate of step i) under suitable conditions to give a compound of formula (I):

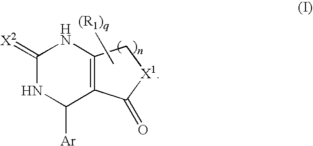

In certain embodiments, the intermediate of step i) has the structure:

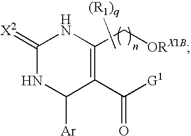

wherein R^{X1B} is an oxygen protecting group, and step ii) comprises steps of:

a) deprotecting the intermediate of step i) under suitable conditions to give an alcohol having the structure:

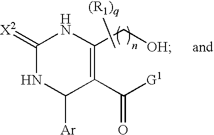

b) reacting the alcohol of step (a) under suitable conditions to give the compound of formula (I^{A}):

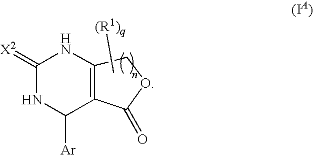

In certain embodiments, R^{X1B} is alkyl, alkenyl, alkynyl, heteroalkyl, heteroalkenyl, heteroalkynyl, aryl, heteroaryl, silyl, —C(═O)R^{x}, —C(═S)R^{x}, C(═NR^{x})R^{y}, —SO₂R^{x}, wherein R^{x} and R^{Y} are each independently hydrogen, alkyl, alkenyl, alkynyl, cycloalkyl, cycloalkenyl, cycloalkynyl, heteroalkyl, heteroalkenyl, heteroalkynyl, heterocycloalkyl, heterocycloalkenyl, heterocycloalkynyl, heteroaliphatic, heteroalicyclic, aryl, heteroaryl, —C(═O)R^{A} or —ZR^{A}, wherein Z is —O—, —S—, —NR^{B}, wherein each occurrence of R^{A} and R^{B} is independently hydrogen, or an alkyl, alkenyl, alkynyl, cycloalkyl, cycloalkenyl, cycloalkynyl, heteroalkyl, heteroalkenyl, heteroalkynyl, heterocycloalkyl, heterocycloalkenyl, heterocycloalkynyl, heteroaliphatic, heteroalicyclic, aryl or heteroaryl moiety. In certain exemplary embodiments, R^{X1B} is a silyl protecting group. In certain exemplary embodiments, R^{X1B} is a trialkylsilyl protecting group. In certain exemplary embodiments, R^{X1B} is triisopropylsilyl.

In certain other embodiments, the intermediate of step i) has the structure:

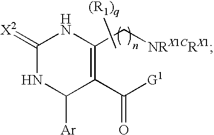

wherein R^{X1C} is hydrogen or a nitrogen protecting group and R^{X1} is hydrogen, alkyl, alkenyl, alkynyl, heteroalkyl, heteroalkenyl, heteroalkynyl, cycloalkyl, heterocycle, acyl, aryl, heteroaryl, -(alkyl)cycloalkyl, -(alkyl)heterocycle, -(heteroalkyl)cycloalkyl, -(heteroalkyl)heterocycle, -(alkyl)aryl, -(heteroalkyl)aryl, -(alkyl)heteroaryl or -(heteroalkyl)heteroaryl;

and step ii) comprises steps of:

a) deprotecting the intermediate of step i) under suitable conditions to give an amino compound having the structure:

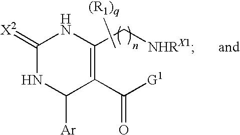

b) reacting the amino compound of step (a) under suitable conditions to give the compound of formula (I^{B}):

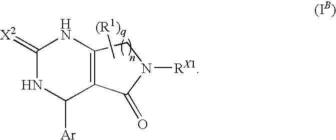

In certain embodiments, R^{X1C} is alkyl, alkenyl, —C(═O)R^{x}, —C(═O)OR^{x}, —SR^{x}, SO₂R^{x}, or R^{X1} and R^{X1C} taken together form a moiety having the structure ═CR^{x}R^{y}, wherein R^{X1} and R^{X1C} are not simultaneously hydrogen and R^{x} and R^{y} are each independently hydrogen, alkyl, alkenyl, alkynyl, cycloalkyl, cycloalkenyl, cycloalkynyl, heteroalkyl, heteroalkenyl, heteroalkynyl, heterocycloalkyl, heterocycloalkenyl, heterocycloalkynyl, heteroaliphatic, heteroalicyclic, aryl, heteroaryl, —C(═O)R^{A} or —ZR^{A}, wherein Z is —O—, —S—, —NR^{B}, wherein each occurrence of R^{A} and R^{B} is independently hydrogen, or an alkyl, alkenyl, alkynyl, cycloalkyl, cycloalkenyl, cycloalkynyl, heteroalkyl, heteroalkenyl, heteroalkynyl, heterocycloalkyl, heterocycloalkenyl, heterocycloalkynyl, heteroaliphatic, heteroalicyclic, aryl or heteroaryl moiety. In certain exemplary embodiments, R^{X1} is hydrogen and R^{X1C} is —C(═O)R^{x}, wherein R^{x} is substituted or unsubstituted lower alkyl or lower alkoxy. In certain exemplary embodiments, R^{X1} is hydrogen and R^{X1C} is —C(═O)R^{x}, wherein R^{x} is tert-Butoxy.

Diversification:

It will also be appreciated that each of the components used in the synthesis of inventive compounds can be diversified either before synthesis or alternatively after the construction of the core structure of formula (I). As used herein, the term “diversifying” or “diversify” means reacting an inventive compound (I) or any of the precursor fragments (or any classes or subclasses thereof) at one or more reactive sites to modify a functional moiety or to add a functional moiety (e.g., nucleophilic addition of a substrate). Described generally herein are a variety of schemes to assist the reader in the synthesis of a variety of compounds, either by diversification of the intermediate components or by diversification of the core structures as described herein, and classes and subclasses thereof. It will be appreciated that a variety of diversification reactions can be employed to generate compounds other than those described in the Exemplification herein. As but a few examples, where a double bond is present in the compound structure, epoxidation and aziridation can be conducted to generate epoxide and aziridine derivatives of compounds described herein. For additional guidance available in the art, the practitioner is directed to “Advanced Organic Chemistry”, March, J. John Wiley & Sons, 2001, 5^{th} ed., the entire contents of which are hereby incorporated by reference.

3) Pharmaceutical Compositions

As discussed above, the present invention provides novel compounds having Eg5 inhibitory activity, and thus the inventive compounds have antitumor and antiproliferative activity and are useful for the treatment of cancer.

Accordingly, in another aspect of the present invention, pharmaceutical compositions are provided, which comprise any one or more of the compounds described herein (or a prodrug, pharmaceutically acceptable salt or other pharmaceutically acceptable derivative thereof), and optionally comprise a pharmaceutically acceptable carrier. In certain embodiments, these compositions optionally further comprise one or more additional therapeutic agents. Alternatively, a compound of this invention may be administered to a patient in need thereof in combination with the administration of one or more other therapeutic agents. For example, additional therapeutic agents for conjoint administration or inclusion in a pharmaceutical composition with a compound of this invention may be an approved chemotherapeutic agent, or it may be any one of a number of agents undergoing approval in the Food and Drug Administration that ultimately obtain approval for the treatment of cancer. In certain other embodiments, the additional therapeutic agent is an anticancer agent, as discussed in more detail herein. In certain other embodiments, the compositions of the invention are useful for the treatment of cancer.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a pro-drug or other adduct or derivative of a compound of this invention which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moeity advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogenfree water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension or crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include (poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose and starch. Such dosage forms may also comprise, as in normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The present invention encompasses pharmaceutically acceptable topical formulations of inventive compounds. The term “pharmaceutically acceptable topical formulation”, as used herein, means any formulation which is pharmaceutically acceptable for intradermal administration of a compound of the invention by application of the formulation to the epidermis. In certain embodiments of the invention, the topical formulation comprises a carrier system. Pharmaceutically effective carriers include, but are not limited to, solvents (e.g., alcohols, poly alcohols, water), creams, lotions, ointments, oils, plasters, liposomes, powders, emulsions, microemulsions, and buffered solutions (e.g., hypotonic or buffered saline) or any other carrier known in the art for topically administering pharmaceuticals. A more complete listing of art-known carriers is provided by reference texts that are standard in the art, for example, Remington's Pharmaceutical Sciences, 16th Edition, 1980 and 17th Edition, 1985, both published by Mack Publishing Company, Easton, Pa., the disclosures of which are incorporated herein by reference in their entireties. In certain other embodiments, the topical formulations of the invention may comprise excipients. Any pharmaceutically acceptable excipient known in the art may be used to prepare the inventive pharmaceutically acceptable topical formulations. Examples of excipients that can be included in the topical formulations of the invention include, but are not limited to, preservatives, antioxidants, moisturizers, emollients, buffering agents, solubilizing agents, other penetration agents, skin protectants, surfactants, and propellants, and/or additional therapeutic agents used in combination to the inventive compound. Suitable preservatives include, but are not limited to, alcohols, quaternary amines, organic acids, parabens, and phenols. Suitable antioxidants include, but are not limited to, ascorbic acid and its esters, sodium bisulfite, butylated hydroxytoluene, butylated hydroxyanisole, tocopherols, and chelating agents like EDTA and citric acid. Suitable moisturizers include, but are not limited to, glycerine, sorbitol, polyethylene glycols, urea, and propylene glycol. Suitable buffering agents for use with the invention include, but are not limited to, citric, hydrochloric, and lactic acid buffers. Suitable solubilizing agents include, but are not limited to, quaternary ammonium chlorides, cyclodextrins, benzyl benzoate, lecithin, and polysorbates. Suitable skin protectants that can be used in the topical formulations of the invention include, but are not limited to, vitamin E oil, allatoin, dimethicone, glycerin, petrolatum, and zinc oxide.

In certain embodiments, the pharmaceutically acceptable topical formulations of the invention comprise at least a compound of the invention and a penetration enhancing agent. The choice of topical formulation will depend or several factors, including the condition to be treated, the physicochemical characteristics of the inventive compound and other excipients present, their stability in the formulation, available manufacturing equipment, and costs constraints. As used herein the term “penetration enhancing agent” means an agent capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). In certain exemplary embodiments, penetration agents for use with the invention include, but are not limited to, triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate) and N-methylpyrrolidone.

In certain embodiments, the compositions may be in the form of ointments, pastes, creams, lotions, gels, powders, solutions, sprays, inhalants or patches. In certain exemplary embodiments, formulations of the compositions according to the invention are creams, which may further contain saturated or unsaturated fatty acids such as stearic acid, palmitic acid, oleic acid, palmito-oleic acid, cetyl or oleyl alcohols, stearic acid being particularly preferred. Creams of the invention may also contain a non-ionic surfactant, for example, polyoxy-40-stearate. In certain embodiments, the active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms are made by dissolving or dispensing the compound in the proper medium. As discussed above, penetration enhancing agents can also be used to increase the flux of the compound across the skin. The rate can be controlled by either providing a rate controlling membrane or by dispersing the compound in a polymer matrix or gel.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anticancer agent), or they may achieve different effects (e.g., control of any adverse effects).

For example, other therapies or anticancer agents that may be used in combination with the inventive compounds of the present invention include surgery, radiotherapy (in but a few examples, γ-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. For a more comprehensive discussion of updated cancer therapies see, The Merck Manual, Seventeenth Ed. 1999, the entire contents of which are hereby incorporated by reference. See also the National Cancer Institute (CNI) website (www.nci.nih.gov) and the Food and Drug Administration (FDA) website for a list of the FDA approved oncology drugs (www.fda.gov/cder/cancer/druglistframe.htm—See Appendix A).

In certain embodiments, the pharmaceutical compositions of the present invention further comprise one or more additional therapeutically active ingredients (e.g., chemotherapeutic and/or palliative). For purposes of the invention, the term “Palliative” refers to treatment that is focused on the relief of symptoms of a disease and/or side effects of a therapeutic regimen, but is not curative. For example, palliative treatment encompasses painkillers, antinausea medications and anti-sickness drugs. In addition, chemotherapy, radiotherapy and surgery can all be used palliatively (that is, to reduce symptoms without going for cure; e.g., for shrinking tumors and reducing pressure, bleeding, pain and other symptoms of cancer).

Additionally, the present invention provides pharmaceutically acceptable derivatives of the inventive compounds, and methods of treating a subject using these compounds, pharmaceutical compositions thereof, or either of these in combination with one or more additional therapeutic agents.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a prodrug or other adduct or derivative of a compound of this invention which upon administration to a patient in need is capable of providing, directly or indirectly, a compound as otherwise described herein, or a metabolite or residue thereof.

4)Research Uses, Pharmaceutical Uses and Methods of Treatment

Research Uses

According to the present invention, the inventive compounds may be assayed in any of the available assays known in the art for identifying compounds having Eg5 inhibitory and/or antiproliferative activity. For example, the assay may be cellular or non-cellular, in vivo or in vitro, high- or low-throughput format, etc.

Thus, in one aspect, compounds of this invention which are of particular interest include those which:
exhibit Eg5-inhibitory activity;
exhibit cytotoxic or growth inhibitory effect on cancer cell lines maintained in vitro or in animal studies using a scientifically acceptable cancer cell xenograft model; and/or
exhibit a therapeutic profile (e.g., optimum safety and curative effect) that is superior to existing chemotherapeutic agents.

As detailed in the exemplification herein, in assays to determine the ability of compounds to inhibit Eg5 activity in purified protein assays, certain inventive compounds exhibited IC₅₀ values≦about 100 μM. In certain embodiments, certain inventive compounds exhibited IC₅₀ values 5 about 50 μM. In certain embodiments, certain inventive compounds exhibited IC₅₀ values≦about 40 μM. In certain embodiments, certain inventive compounds exhibited IC₅₀ values≦about 30 μM. In certain embodiments, certain inventive compounds exhibited IC₅₀ values≦about 20 μM. In certain embodiments, certain inventive compounds exhibited IC₅₀ values≦about 10 μM. In certain embodiments, certain inventive compounds exhibited IC₅₀ values≦about 5 μM. In certain embodiments, certain inventive compounds exhibited IC₅₀ values≦about 4 μM. In certain embodiments, certain inventive compounds exhibited IC₅₀ values≦about 3 μM. In certain embodiments, certain inventive compounds exhibited IC₅₀ values≦about 2.5 μM. In certain embodiments, certain inventive compounds exhibited IC₅₀ values≦about 2 μM. In certain embodiments, certain inventive compounds exhibited IC₅₀ values≦about 1.5 μM. In certain embodiments, certain inventive compounds exhibited IC₅₀ values≦about 1 μM. In certain embodiments, certain inventive compounds exhibit IC₅₀ values 5 about 500 nM. In certain embodiments, certain inventive compounds exhibit IC₅₀ values 5 about 250 nM. In certain embodiments, certain inventive compounds exhibit IC₅₀ values 5 about 100 nM. In certain embodiments, certain inventive compounds exhibit IC₅₀ values≦about 50 nM.

As detailed in the exemplification herein, in assays to determine the ability of compounds to inhibit Eg5 activity in cells, certain inventive compounds exhibited IC₅₀ values≦about 500 M. In certain embodiments, certain inventive compounds exhibited IC₅₀ values≦about 400 μM. In certain embodiments, certain inventive compounds exhibited IC₅₀ values≦about 350 μM. In certain embodiments, certain inventive compounds exhibited IC₅₀ values≦about 300 μM. In certain embodiments, certain inventive compounds exhibited IC₅₀ values≦about 250 μM. In certain embodiments, certain inventive compounds exhibited IC₅₀ values≦about 200 μM. In certain embodiments, certain inventive compounds exhibited IC₅₀ values≦about 150 μM. In certain embodiments, certain inventive compounds exhibited IC₅₀ values≦about 100 μM. In certain embodiments, certain inventive compounds exhibited IC₅₀ values≦about 75 μM. In certain embodiments, certain inventive compounds exhibited IC₅₀ values≦about 50 μM. In certain embodiments, certain inventive compounds exhibited IC₅₀ values≦about 40 μM. In certain embodiments, certain inventive compounds exhibited IC₅₀ values≦about 30 μM. In certain embodiments, certain inventive compounds exhibit IC₅₀ values 5 about 25 μM. In certain embodiments, certain inventive compounds exhibit IC₅₀ values≦about 20 μM. In certain embodiments, certain inventive compounds exhibit IC₅₀ values≦about 15 μM. In certain embodiments, certain inventive compounds exhibit IC₅₀ values≦about 10 μM. In certain embodiments, certain inventive compounds exhibit IC₅₀ values≦about 5 μM. In certain embodiments, certain inventive compounds exhibit IC₅₀ values≦about 2.5 μM. In. certain embodiments, certain inventive compounds exhibit IC₅₀ values≦about 1 μM.

Pharmaceutical Uses and Methods of Treatment

In general, methods of using the compounds of the present invention comprise administering to a subject in need thereof a therapeutically effective amount of a compound of the present invention. As discussed above, compounds of the invention are inhibitors of Eg5 and, as such, are useful in the treatment of disorders in which Eg5 is involved. For example, compounds of the invention may be useful in the treatment of cancer. Accordingly, in yet another aspect, according to the methods of treatment of the present invention, tumor cells are killed, or their growth is inhibited by contacting said tumor cells with an inventive compound or composition, as described herein.

Thus, in another aspect of the invention, methods for the treatment of cancer are provided comprising administering a therapeutically effective amount of a compound of formula (I), as described herein, to a subject in need thereof. In certain embodiments, a method for the treatment of cancer is provided comprising administering a therapeutically effective amount of an inventive compound, or a pharmaceutical composition comprising an inventive compound to a subject in need thereof, in such amounts and for such time as is necessary to achieve the desired result. In certain embodiments of the present invention a “therapeutically effective amount” of the inventive compound or pharmaceutical composition is that amount effective for killing or inhibiting the growth of tumor cells. The compounds and compositions, according to the method of the present invention, may be administered using any amount and any route of administration effective for killing or inhibiting the growth of tumor cells. Thus, the expression “amount effective to kill or inhibit the growth of tumor cells”, as used herein, refers to a sufficient amount of agent to kill or inhibit the growth of tumor cells. The exact amount required will vary from subject to subject, depending on the species, age, and general condition of the subject, the severity of the infection, the particular anticancer agent, its mode of administration, and the like.

In certain embodiments, any system where the control of cellular growth and cell division is desired may utilize compounds in the bicyclic dihydropyrimidine class to regulate mitosis. More specifically, invnetive compounds may be used to inhibit cell growth. One non-limiting example of an application of compounds of the invention to a cellular system is their use as a anti-mitotic anti-cancer drugs. Other examples include controlling cell division and the immune system in diseases such as rheumatoid arthritis

In certain embodiments, a method of treating an individual with uncontrolled or abnormal cell growth is provided. Compositions comprising one or more inventive compounds or derivatives thereof with similar biological activity are useful for treating individuals with cells that having become cancerous tumors. Such compositions may be administered to an individual in need thereof at therapeutically effective amounts to slow or cease the abnormal cell growth. Generally, abnormal cell growth is associated with cancerous cells. However, other diseases resulting from uncontrolled cell growth (e.g. cardiovascular diseases, rheumatoid arthritis etc.) may be treated with compositions and methods of the present invention.

In certain embodiments, the method involves the administration of a therapeutically effective amount of the compound or a pharmaceutically acceptable derivative thereof to a subject (including, but not limited to a human or animal) in need of it.

As discussed above, the compounds of the present invention are useful as anticancer agents, and thus may be useful in the treatment of cancer, by effecting tumor cell death or inhibiting the growth of tumor cells. In certain embodiments, the inventive compounds as useful for the treatment of cancer (including, but not limited to, glioblastoma, retinoblastoma, breast cancer, cervical cancer, colon and rectal cancer, leukemia, lymphoma, lung cancer (including, but not limited to small cell lung cancer), melanoma and/or skin cancer, multiple myeloma, non-Hodgkin's lymphoma, ovarian cancer, pancreatic cancer, prostate cancer and gastric cancer, bladder cancer, uterine cancer, kidney cancer, testicular cancer, stomach cancer, brain cancer, liver cancer, or esophageal cancer). In certain embodiments, the inventive anticancer agents are active against leukemia cells and melanoma cells, and thus are useful for the treatment of leukemias (e.g., myeloid, lymphocytic, myelocytic and lymphoblastic leukemias) and malignant melanomas. In still other embodiments, the inventive anticancer agents kill and/or inhibit the growth of multidrug resistant cells (MDR cells). In still other embodiments, the inventive anticancer agents are active against solid tumors.

Additionally, the present invention provides pharmaceutically acceptable derivatives of the inventive compounds, and methods of treating a subject using these compounds, pharmaceutical compositions thereof, or either of these in combination with one or more additional therapeutic agents.

Another aspect of the invention relates to a method of treating or lessening the severity of a disease or condition associated with a proliferation disorder in a patient, said method comprising a step of administering to said patient, a compound of formula I or a composition comprising said compound.

It will be appreciated that the compounds and compositions, according to the method of the present invention, may be administered using any amount and any route of administration effective for the treatment of cancer and/or disorders associated with cell hyperproliferation. For example, when using the inventive compounds for the treatment of cancer, the expression “effective amount” as used herein, refers to a sufficient amount of agent to inhibit cell proliferation, or refers to a sufficient amount to reduce the effects of cancer. The exact amount required will vary from subject to subject, depending on the species, age, and general condition of the subject, the severity of the diseases, the particular anticancer agent, its mode of administration, and the like.

The compounds of the invention are preferably formulated in dosage unit form for ease of administration and uniformity of dosage. The expression “dosage unit form” as used herein refers to a physically discrete unit of therapeutic agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts (see, for example, Goodman and Gilman's, “The Pharmacological Basis of Therapeutics”, Tenth Edition, A. Gilman, J. Hardman and L. Limbird, eds., McGraw-Hill Press, 155-173, 2001, which is incorporated herein by reference in its entirety).

Another aspect of the invention relates to a method for inhibiting Eg5 activity in a biological sample or a patient, which method comprises administering to the patient, or contacting said biological sample with at least one compound of formula I or a composition comprising said compound.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, creams or drops), bucally, as an oral or nasal spray, or the like, depending on the severity of the infection being treated. In certain embodiments, the compounds of the invention may be administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. In certain embodiments, compounds are administered orally or parenterally.

As discussed above, the present invention provides a method of treating a condition via modulation of the Eg5 protein activity comprising administering to a subject in need thereof an effective amount at least one compound of the invention. The invention also provides a method for treating a condition via modulation of the Eg5 protein activity comprising administering to a subject in need thereof a combination (simultaneous or sequential) of at least one antineoplastic agent and at least one compound of the invention. In a preferred embodiment, the condition treated is a proliferative disease such as cancer. Any compounds that act as antineoplastic agents and any small molecule which modulates the Eg5 protein sufficiently to induce mitotic arrest and apoptosis can be used in the instant invention. Monastrol has not been shown to induce apoptosis and is not included within the scope of this invention.

When combination therapy is employed, it is anticipated that the therapeutic effect of the instant invention may be achieved with smaller amounts of the antineoplastic agents and Eg5 protein inhibitors than would be required if such antineoplastic agents and Eg5 inhibitors were administered alone, thereby avoiding or minimizing adverse toxicity effects.

Antineoplastic agents which are suitable for use in the methods and compositions of this invention include, but are not limited to, microtuble-stabilizing agents such as paclitaxel (also known as Taxol®), docetaxel (also known as Taxotere®), 7-O-methylthiomethylpaclitaxel (disclosed in U.S. Pat. No. 5,646,176, herein incorporated by reference), 3′-tert-butyl-3′-N-tert-butyloxycarbon-yl-4-deacetyl-3′-dephenyl-3′-N-debenzoyl-4-O-methoxycarbonyl-paclitaxel, C-4 methyl carbonate paclitaxel (disclosed in WO 94/14787, herein incorporated by reference), epothilone A, epothilone B, epothilone C, epothilone D, desoxyepothilone A, desoxyepothilone B, [1S-[1R*,3R*(E),7R*,10S*,11R*,12R*,16S*]]-7-11-dihydroxy-8,8,10,12,16-pentamethyl-3-[1-methyl-2-(2-methyl-4-thiazolyl)ethenyl]-4-aza-17-oxabicyclo[14.1.0]heptadecane-5,9-dione (disclosed in WO 99/02514, herein incorporated by reference), [1S-[1R*,3R*(E), 7R*,10S*,11R*,12R*,16S*]]-3-[2-[2-(aminomethyl)-4-thiazolyl]-1-methylethenyl]-7,11-dihydroxy-8,8,10,12,16-pentamethyl-4-17-dioxabicyclo-[14.1.0]-heptadecane-5,9-dione, and derivatives thereof; microtuble-disruptor agents; inhibitors of cyclin dependent kinases (including those disclosed in U.S. Pat. No. 6,040,321, herein incorporated by reference); inhibitors of farnesyltransferase; alkylating agents; anti-metabolites; epidophyllotoxin; an antineoplastic enzyme; a topoisomerase inhibitor; procarbazine; mitoxantrone; platinum coordination complexes; biological response modifiers; growth factor inhibitors; hormonal/antihormonal therapeutic agents; and haematopoietic growth factors.

Classes of antineoplastic agents suitable for use in the present invention include, but are not limited to, the anthracycline family of drugs, the vinca drugs, the mitomycins, the bleomycins, the cytotoxic nucleosides, the taxanes, the epothilones, discodermolide, the pteridine family of drugs, diynenes, aromatase inhibitors, and the podophyllotoxins. Particularly useful members of those classes include, for example, paclitaxel, docetaxel, 7-O-methylthiomethylpacliitaxel, 3′-tert-butyl-3′-N-tert-butyloxycarbonyl-4-deacetyl-3′-dephenyl-3′-N-debe-nzoyl-4-O-methoxycarbonyl-paclitaxel, C-4 methyl carbonate paclitaxel, epothilone A, epothilone B, epothilone C, epothilone D, desoxyepothilone A, desoxyepothilone B, [1S-[1R*,3R*(E),7R*,10S*,11R*, 12R*,16S*]]-7,11-dihydroxy-8,8,10,12,16-pentamethyl-3-[1-methyl-2-(2-methyl-4-thiazolyl)ethenyl]-4-aza-17-oxabicyclo[14.1.0]heptadecane-5,9-dione, [1S-[1R*,3R*(E),7R*,10S*,11R*,12R*,16S*]]-3-[2-[2-(aminomethyl)-4-thiazolyl]-1-methylethenyl]-7,11-dihydroxy-8,8,10,12,16-pentamethyl-4,17-dioxabicyclo[14.1.0]-heptadecane-5,9-dione, doxorubicin, caminomycin, daunorubicin, aminopterin, methotrexate, methopterin, dichloro-methotrexate, mitomycin C, porfiromycin, 5-fluorouriacil, 6-mercaptopurine, gemcitabine, cytosine arabinoside, podophyllotoxin or podophyllotoxin derivatives such as etoposide, etoposide phosphate or teniposide, melphalan, vinblastine, vincristine, leurosidine, vindesine, leurosine, and the like. Other useful antineoplastic agents include estramustine, cisplatin, carboplatin, cyclophosphamide, bleomycin, tamoxifen, ifosamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, pyridobenzoindole derivatives, interferons, interleukins, and inhibitors of cyclin dependent kinases including, but not limited to, those in U.S. Pat. No. 6,040,321, herein incorporated by reference; and inhibitors of farnesyltransferase including, but not limited to, those in U.S. Pat. No. 6,011,029 herein incorporated by reference.

Preferred classes of antineoplastic agents are the taxanes and the epothilones, and the preferred antineoplastic agents are paclitaxel, docetaxel, 7-O-methylthio-methylpaclitaxel, 3′-tert-butyl-3′-N-tert-butyl-oxycarbonyl-4-deacetyl-3′-dephenyl-3′-N-debenzoyl-4-O-methoxycarbonyl-pacl-itaxel, C-4 methyl carbonate paclitaxel, epothilone A, epothilone B, epothilone C, epothilone D, desoxyepothilone A, desoxyepothilone B, [1S-[1R*,3R*(E),7R*,10S*,11R*,12R*,16S*]]-7,11-dihydroxy-8,8,10,12,16-pentamethyl-3-[1-methyl-2-(2-methyl-4-thiazolyl-)ethenyl]-4-aza-17-oxabicyclo[14.1.0]heptadecane-5,9-dione, and [1S-[1R*,3R*(E), 7R*, 100S*,11R*, 12R*,16S*]]-3-[2-[2-(aminomethyl)-4-thiazolyl]-1-methylethenyl]-7,11-dihydroxy-8,8,10,12,16-pentamethyl-4,17-dioxabicycl-o[14.1.0]heptadecane-5,9-dione, the cyclin dependent kinase exemplified in U.S. Pat. No. 6,040,321; the farnesyltransferase inhibitors exemplified in U.S. Pat. No. 6,011,029 as well as (R)-7-cyano-2,3,4,5-tetrahydro-1-(1H-imidazol-4-ylmethyl)-3-(phenylmethyl)-4-(2-thienylsulfonyl)-1H-1,4-benzodiazepine, mesylate salt.

### Treatment Kit

In other embodiments, the present invention relates to a kit for conveniently and effectively carrying out the methods in accordance with the present invention. In general, the pharmaceutical pack or kit comprises one or more containers filled with one or more of the ingredients of the pharmaceutical compositions of the invention. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the dosages of the pharmaceutical compositions, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that, unless otherwise indicated, the entire contents of each of the references cited herein are incorporated herein by reference to help illustrate the state of the art. The following examples contain important additional information, exemplification and guidance which can be adapted to the practice of this invention in its various embodiments and the equivalents thereof.

These and other aspects of the present invention will be further appreciated upon consideration of the following Examples, which are intended to illustrate certain particular embodiments of the invention but are not intended to limit its scope, as defined by the claims.

### EXEMPLIFICATION

The compounds of this invention and their preparation can be understood further by the examples that illustrate some of the processes by which these compounds are prepared or used. It will be appreciated, however, that these examples do not limit the invention. Variations of the invention, now known or further developed, are considered to fall within the scope of the present invention as described herein and as hereinafter claimed.

Two novel classes of constrained analogs of S-Monastrol—a known Eg5 inhibitor—were conceived, species of which are depicted below:

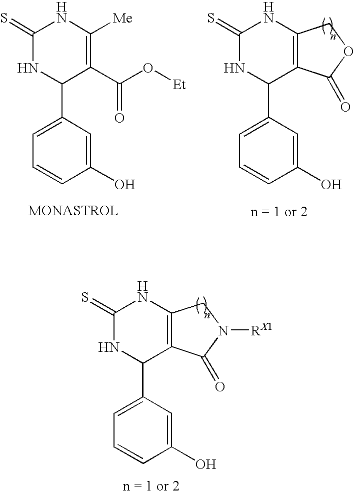

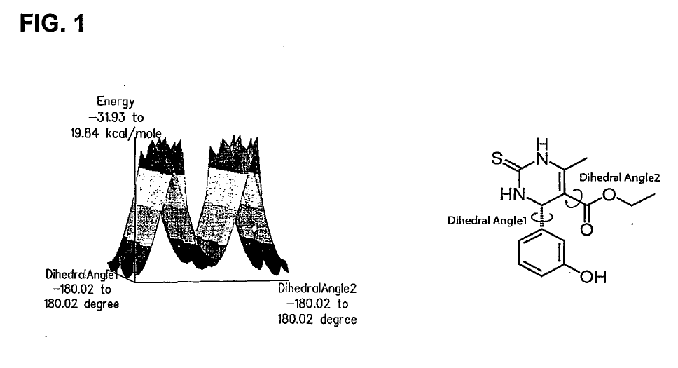

The assumption that this class of compounds could be more potent Eg5 inhibitors than Monastrol itself was made based on semi-empirical quantum mechanics calculation (PM3). These calculations were performed in order to get insight in the potential energies of individual conformations of Monastrol. Only the rotatable bonds of the phenilc substituent and the ethyl ester side chain were selected as search parameters. Results are shown in FIG. 1.

We found that the energies between the s-cis and the s-trans conformations of the ester side chain differ by 2.4 to 2.9 kcal/mol in favor of the s-trans conformation.

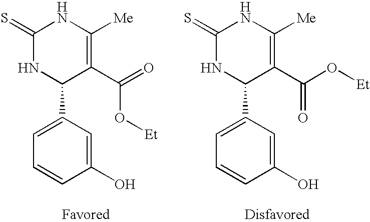

This results in a population ratio os 1:40 to 1:100 for the two individual conformers. Assuming that the disfavored conformation is the biologically active species, an increase of the potency by up to two orders of magnitude seemed feasible if the molecule was arrested in this state.

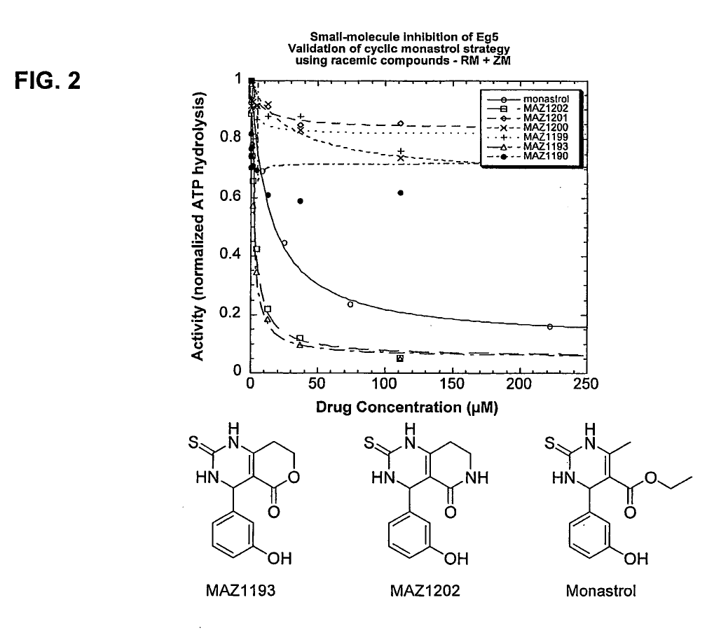

Compounds were tested and showed a highly improved activity towards the isolated Eg5 protein. The IC50 values were more than one order of magnitude lower than that of Monastrol (See FIG. 2).

Furthermore, it was shown that all tested compounds retained their activity in cell based assays. Additionally, the tested compounds showed an improved solubility profile as compared to Monastrol.

1) General Description of Synthetic Methods:

Additional synthetic guidance is provided elsewhere in this document.

The compounds of this invention and their preparation can be understood further by the examples that illustrate some of the processes by which these compounds are prepared or used. It will be appreciated, however, that these examples do not limit the invention. Variations of the invention, now known or further developed, are considered to fall within the scope of the present invention as described herein and as hereinafter claimed.

According to the present invention, any available techniques can be used to make or prepare the inventive compounds or compositions including them. For example, a variety of solution phase synthetic methods such as those discussed in detail below may be used. Alternatively or additionally, the inventive compounds may be prepared using any of a variety of combinatorial techniques, parallel synthesis and/or solid phase synthetic methods known in the art.

It will be appreciated as described below, that a variety of inventive compounds can be synthesized according to the methods described herein. The starting materials and reagents used in preparing these compounds are either available from commercial suppliers such as Aldrich Chemical Company (Milwaukee, Wis.), Bachem (Torrance, Calif.), Sigma (St. Louis, Mo.), or are prepared by methods well known to a person of ordinary skill in the art following procedures described in such references as Fieser and Fieser 1991, “Reagents for Organic Synthesis”, vols 1-17, John Wiley and Sons, New York, N.Y., 1991; Rodd 1989 “Chemistry of Carbon Compounds”, vols. 1-5 and supps, Elsevier Science Publishers, 1989; “Organic Reactions”, vols 1-40, John Wiley and Sons, New York, N.Y., 1991; March 2001, “Advanced Organic Chemistry”, 5th ed. John Wiley and Sons, New York, N.Y.; and Larock 1990, “Comprehensive Organic Transformations: A Guide to Functional Group Preparations”, 2^{nd} ed. VCH Publishers. These schemes are merely illustrative of some methods by which the compounds of this invention can be synthesized, and various modifications to these schemes can be made and will be suggested to a person of ordinary skill in the art having regard to this disclosure.

The starting materials, intermediates, and compounds of this invention may be isolated and purified using conventional techniques, including filtration, distillation, crystallization, chromatography, and the like. They may be characterized using conventional methods, including physical constants and spectral data.

1-a) Synthesis of Exemplary Compounds:

Unless otherwise indicated, starting materials are either commercially available or readily accessibly through laboratory synthesis by anyone reasonably familiar with the art. Described generally below, are procedures and general guidance for the synthesis of compounds as described generally and in subclasses and species herein. Unless mentioned specifically, reaction mixtures were stirred using a magnetically driven stirrer bar. An inert atmosphere refers to either dry argon or dry nitrogen. Reactions were monitored either by thin layer chromatography, by proton nuclear magnetic resonance or by high-pressure liquid chromatography (HPLC), of a suitably worked up sample of the reaction mixture.

An exemplary synthetic approach to Cyclic-Monastrol derivatives O-series is depicted in Scheme 4.

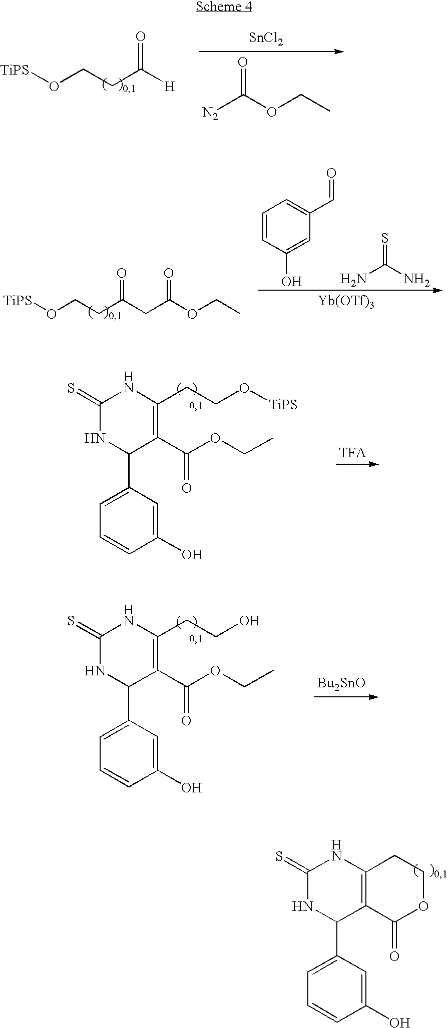

An exemplary synthetic approach to Cyclic-Monastrol derivatives N-series is depicted in Scheme 5.

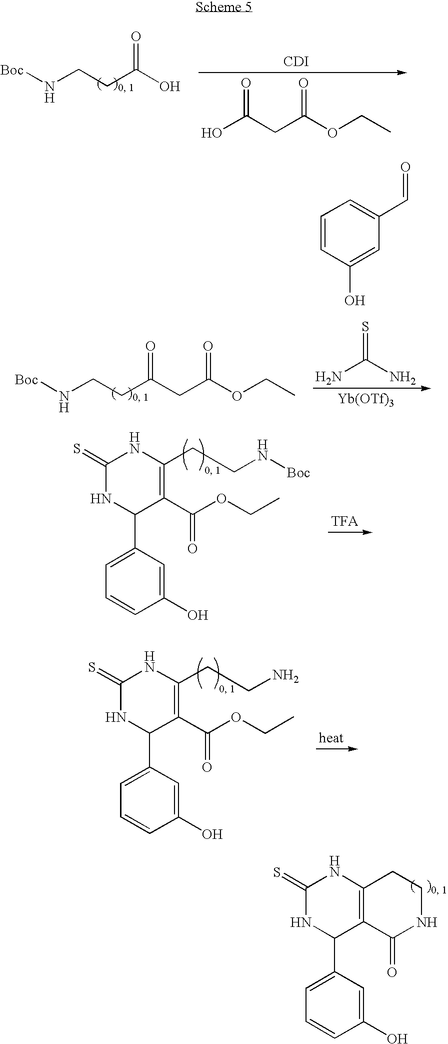

### Example 1

### 3-Oxo-4-triisopropylsilanyloxy-butyric acid ethyl ester

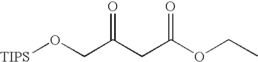

3.24 g (15 mmol) TIPS-protected Hydroxy-acetaldehyde and 284 mg (1.5 mmol) powdered tin (II) chloride were suspended in 40 ml Methylene Chloride and placed in a 30° C. water bath. 2.56 g (22.5 mmol) Ethyl diazoacetate were added via syringe over 5 min and the yellow suspension was stirred for 30 min. Water and 1M HCl were added to the solution and the aqueous layer was extracted three times with ether. The combined organic layers were dried over Na₂SO₄. The solvent was removed under reduced pressure and the crude product was purified on silica (Ether/Hexanes 4:1) to yield the desired ketoester (3.69 g, 82%) as a colorless oil.

¹H-NMR (500 MHz, CDCl₃); 4.29 (s, 2H); 4.18 (q, 2H, J=7.1 Hz); 3.61 (s, 2H); 1.27 (t, 3H, J=7.1 Hz); 1.14 (m, 3H); 1.06 (m, 18H)

### Example 2

### 3-Oxo-5-triisopropylsilanyloxy-pentanoic acid ethyl ester

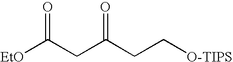

Prepared according to literature reference (D. J. Kopecky and S. D. Rychnovsky, J. Am. Chem. Soc. 2001, 123, 8240-8421)

### Example 3

### 5-tert-Butoxycarbonylamino-3-oxo-pentanoic acid ethyl ester

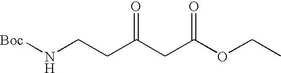

1,1′-carbonyldiimidazole (12.15, 76 mmol) was added to a solution N-Boc beta-Alanine (14.3 g 76 mmol) in dry THF (100 ml) and stirred under nitrogen for 2 h to form the acid imidazolide.

To ethyl hydrogen malonate (10 g, 76 mmol) in dry THF (100 ml) at 0° C. was added 100 ml freshly prepared propan-2-ylmagnesium bromid in THF (144 mmol) over 10 min. After stirring at room temperature for 30 min and warming to 40° C. for 30 min, the solution was cooled to 0° C. and the imidazolide solution added. The resultant gummy precipitate was stirred vigorously at room temperature for 4 h and cooled to 0° C. Saturated bicarbonate solution was added to the solution. The aqueous layer was extracted three times with EtOAc. 20 ml of sat. CuSO4 were added to the organic layer, whereby the organic layer turned green. Subsequently 1M HCl was added in small portions until the blue color remained in the aqueous layer. The organic layer was dried over Na₂SO₄. After evaporation of the solvent under reduced pressure, the crude product was purified on silica (Hexanes/Ethyl Acetate 2:1) to yield the Ketoester as a colorless oil (13.2 g, 51 mmol, 68%).

1H-NMR (500 MHz, CDCl3); 4.96 (s, 1H); 4.19 (q, 2H, J=7.1 Hz); 3.44 (s, 2H); 3.38 (dd, 2H, J=5.0 Hz, J=10.8 Hz); 2.78 (t, 2H, J=5.7 Hz); 1.42 (s, 9H); 1.28 (t, 3H, J=7.1 Hz)

### Example 4

### 4-tert-Butoxycarbonylamino-3-oxo-butyric acid ethyl ester

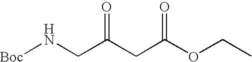

To N-Boc Glycine (6.65 g 38 mmol) in dry THF (50 ml) was added 1,1′-carbonyldiimidazole (6.16 g, 38 mmol) and the reaction was stirred under nitrogen for 2 h to form the acid imidazolide.

To ethyl hydrogen malonate (5 g g, 38 mmol) in dry THF (50 ml) at 0° C. was added 50 ml over 10 min freshly prepared propan-2-ylmagnesium bromid in THF (72 mmol). After stirring at room temperature for 30 min and warming to 40° C. for 30 min, the solution was cooled to 0° C. and the imidazolide solution added. The resultant gummy precipitate was stirred vigorously at room temperature for 4 h, saturated bicarbonate solution was added after cooling to 0° C. The aqueous layer was extracted three times with EtOAc. 20 ml of sat. CuSO4 were added to the organic layer, whereby the organic layer turned green. Subsequently 1M HCl was added in small portions until the blue color remained in the aqueous layer. The organic layer was dried over Na₂SO₄. After evaporation of the solvent under reduced pressure the crude product was purified on silica (Hexanes/Ethyl acetate 2:1) to yield the Ketoester as a colorless oil (5.7 g, 23.2 mmol, 61%).

1H-NMR (500 MHz, CDCl3); 5.18 (s, 1H); 4.19 (q, 2H, J=7.1 Hz); 4.12 (d, 2H, J=4.6 Hz); 3.47 (s, 2H); 1.44 (s, 9H); 1.27 (t, 3H, J=7.2 Hz)

### Example 5

### General Procedure for the Biginelli Reaction

The Ketoester (2 mmol), 3-Hydroxybenzaldehyde (2 mmol), Thiourea (3 mmol, 1.5 eq) and Yb(OTf)₃ (0.2 mmol, 01 eq.) were dissolved in 4 ml Acetonitrile and stirred under inert conditions for 2-24 h at 90° C. After cooling to room temperature, the crude reaction was poured into water and extracted three times with Methylene Chloride. The combined organic layers were dried over Na₂SO₄. After evaporation of the solvent, the crude product was purified on silica.

### Example 6

### 4-(3-Hydroxy-phenyl)-2-thioxo-6-(2-triisopropylsilanyloxy-ethyl)-1,2,3,4-tetrahydro-pyrimidine-5-carboxylic acid ethyl ester

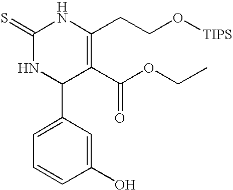

yellow oil (yield 44%), purification with 10-50% Ethyl acetate in Hexanes

1H-NMR (500 MHz, CDCl3); 8.84 (s, 1H); 7.75 (s, 1H); 7.08 (t, 1H, J=7.8 Hz); 6.80 (d, 1H, J=7.8 Hz); 6.7 (br, 1H); 6.75 (m, 1H); 6.72 (dd, 1H, J=2.6 Hz, J=7.6 Hz); 5.29 (d, 1H, J=3.1 Hz); 4.08 (m, 2H); 4.00 (m, 2H); 3.19 (ddd, 1H, J=3.6 Hz, J=6.2 Hz, J=14.9 Hz); 3.02 (ddd, 1H, J=3.9 Hz, J=7.3 Hz, J=15.1 Hz); 1.14 (m, 24H)

HRMS (M+H⁺, calc. 479.2400, found 479.2397)

### Example 7

### 4-(3-Hydroxy-phenyl)-2-thioxo-6-triisopropylsilanyloxymethyl-1,2,3,4-tetrahydro-pyrimidine-5-carboxylic acid ethyl ester

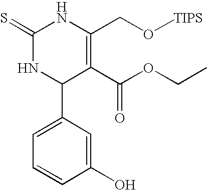

yellow oil (yield 34%), purification with 20-50% Ethyl acetate in Hexanes

1H-NMR (500 MHz, CDCl3); 8.71 (s, 1H); 7.84 (s, 1H); 7.06 (t, 1H, J=7.8 Hz); 6.73 (m, 4H); 5.27 (d, 1H, J=2.4 Hz); 4.91 (q, 2H, J=17.1 Hz); 4.06 (m, 2H); 1.08 (m, 24H).

HRMS (M+H⁺, calc. 465.2243, found 465.2242)

### Example 8

### 6-(2-tert-Butoxycarbonylamino-ethyl)-4-(3-hydroxy-phenyl)-2-thioxo-1,2,3,4-tetrahydro-pyrimidine-5-carboxylic acid ethyl ester

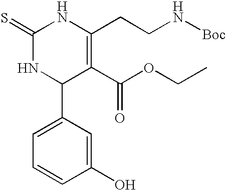

yellow oil (yield 48%), purification with 10-70% Ethyl acetate in Hexanes

1H-NMR (500 MHz, CD₃OD); 7.11 (t, 1H, J=7.8 Hz); 6.75 (ddd, 1H, J=1.0 Hz, J=1.5 Hz, J=7.5 Hz); 6.73 (t, 1H, J=2.2 Hz); 6.69 (ddd, 1H, J=0.9 Hz, J=2.5 Hz, J=8.1 Hz); 5.25 (s, 1H); 4.06 (dq, 2H, J=1.7 Hz, J=7.1 Hz); 3.32 (m, 2H,); 2.88 (m, 2H); 1.42 (s, 9H); 1.15 (t, 3H, J=7.1 Hz)

HRMS (M+H⁺, calc. 422.1749, found 422.1754)

### Example 9

### 6-(tert-Butoxycarbonylamino-methyl)-4-(3-hydroxy-phenyl)-2-thioxo-1,2,3,4-tetrahydro-pyrimidine-5-carboxylic acid ethyl ester

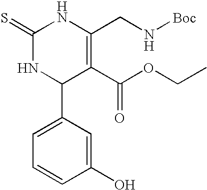

yellow oil (yield 16%), purification with 10-70% Ethyl acetate in Hexanes

1H-NMR (500 MHz, CDCl3); 8.80 (s, 1H); 8.10 (s, 1H,); 7.20 (s, 1H); 7.07 (t, 1H, J=7.9 Hz); 6.80 (br, 1H); 6.73 (m, 3H); 5.24 (s, 1H); 4.38 (m, 2H); 4.06 (q, 2H, J=7.0 Hz); 1.44 (s, 9H); 1.14 (t, 3H, J=7.1 Hz)

HRMS (M+H⁺, calc. 408.1593 found 408.1583)

### Example 10

### General Procedure: Boc-Deprotection

The Boc-protected amine was dissolved in Methylene Chloride (0.2M). After cooling to 0° C. Trifluorocetic acid (5% v/v) was added. The reaction was warmed to room temperature and stirred for additional 4 h, before the solvent was evaporated under reduced pressure and the amine was used for the following cyclization without further purification.

### Example 11

### 6-(2-Amino-ethyl)-4-(3-hydroxy-phenyl)-2-thioxo-1,2,3,4-tetrahydro-pyrimidine-5-carboxylic acid ethyl ester

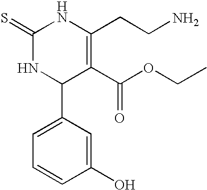

1H-NMR (500 MHz, CD₃OD); 7.14 (t, 1H, J=8.0 Hz); 6.78 (d, 1H, J=7.7 Hz); 6.75 (m, 1H); 6.71 (ddd, 1H, J=0.9 Hz, J=2.2 Hz, J=8.0 Hz); 5.27 (s, 1H); 4.11 (dd, 2H, J=7.0 Hz, J=14.0 Hz); 3.22 (m 3H); 2.94 (td, 1H, J=6.5 Hz, J=7.7 Hz); 1.16 (t, 3H, J=7.1 Hz)

HRMS (M+H⁺, calc. 322.1225, found 322.1232)

### Example 12

### 6-Aminomethyl-4-(3-hydroxy-phenyl)-2-thioxo-1,2,3,4-tetrahydro-pyrimidine-5-carboxylic acid ethyl ester

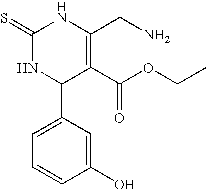

1H-NMR (500 MHz, CD₃OD); 7.16 (t, 1H, J=7.9 Hz); 6.82 (d, 1H, J=7.7 Hz); 6.78 (t, 1H, J=2 Hz); 6.73 (ddd, 1H, J=0.9 Hz, J=2.4 Hz, J=8.1 Hz); 5.32 (s, 1H); 4.19 (m, 2H); 4.14 (d, 1H, J=13.5 Hz); 4.07 (d, 1H, J=13.4 Hz); 1.22 (t, 311, J=7.1 Hz)

HRMS (M+H⁺, calc. 308.1069, found 308.1075)

### Example 13

### General Procedure: TIPS-Deprotection

The TIPS-protected alcohol was dissolved in THF (0.2M). After addition of 15% HF/Pyridine (v/v) the reaction was stirred at room temperature. After 3 h excess HF was quenched by addition of 2 eq. of TMSOEt. The solvent is removed under reduced pressure and the crude product was purified on silica (0-10% Methanol in Methylene Chloride) to yield the desired alcohol as a yellow oil.

### Example 14

### 6-(2-Hydroxy-ethyl)-4-(3-hydroxy-phenyl)-2-thioxo-1,2,3,4-tetrahydro-pyrimidine-5-carboxylic acid ethyl ester

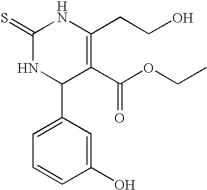

1H-NMR (500 MHz, CD₃OD); 7.13 (t, 1H, J=7.8 Hz); 6.78 (dm, 1H, J=7.7 Hz); 6.74 (t, 1H, J=7.8 Hz); 6.68 (dd, 1H, J=0.9 Hz, J=2.5 Hz); 5.26 (s, 1H); 4.08 (dq, 2H, J=1.9 Hz, J=7.1 Hz); 3.80 (t, 1H, J=6.7 Hz); 3.34 (s, 1H,); 3.02 (m, 2H); 1.17 (t, 3H, J=7.1 Hz)

HRMS (M+H⁺, calc. 323.1065, found 323.1066)

### Example 14

### 6-Hydroxymethyl-4-(3-hydroxy-phenyl)-2-thioxo-1,2,3,4-tetrahydro-pyrimidine-5-carboxylic acid ethyl ester

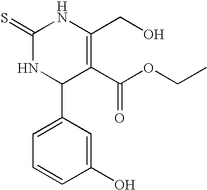

1H-NMR (500 MHz, CD₃OD); 7.13 (t, 1H, J=7.8 Hz); 6.79 (m, 2H); 6.70 (ddd, 1H, J=1.0 Hz, J=2.3 Hz, J=8.1 Hz); 5.28 (s, 1H); 4.77 (d, 1H, J=16.9 Hz); 4.70 (d, 1H, J=16.9 Hz); 4.10 (m, 2H); 1.18 (t, 3H, J=6.9 Hz)

HRMS (M+H⁺, calc. 309.0909, found 309.0903)

### Example 15

### 4-(3-Hydroxy-phenyl)-2-thioxo-1,2,3,4,7,8-hexahydro-pyrano[4,3-d]pyrimidin-5-one

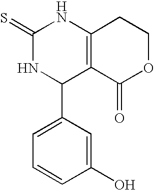

20 mg of the deprotected alcohol and 20 mg Dibutyltinoxide were dissolved in 2 ml Methanol. The reaction mixture was stirred for 16 h at 70° C. The solvent was removed under reduced pressure and the crude product was purified by reversed phase HPLC (AtlantisAQ) to yield 14 mg of the desired product as a white solid (82%).

1H-NMR (500 MHz, CD₃OD); 7.14 (t, 1H, J=7.9 Hz); 6.80 (d, 1H, J=7.6 Hz); 6.69 (ddd, 1H, J=0.9 Hz, J=2.5 Hz, J=8.1 Hz); 5.25 (d, 1H, J=1.1 Hz); 4.41 (ddd, 1H, J=3.3 Hz, J=5.7 Hz, J=11.2 Hz); 4.28 (dt, 1H, J=4.2 Hz, J=11.6 Hz); 2.80 (dddd, 1H, J=1.3 Hz, J=5.7 Hz, J=11.8 Hz, J=17.6 Hz); 2.55 (td, 1H, J=3.8 Hz, J=17.7 Hz)

HRMS (M+H⁺, calc. 277.0647, found 277.0648)

### Example 16

### 4-(3-Hydroxy-phenyl)-2-thioxo-2,3,4,7-tetrahydro-1H-furo[3,4-d]pyrimidin-5-one

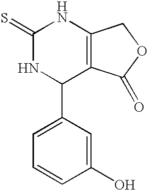

5 mg of the deprotected alcohol and 10 mg Dibutyltinoxide were dissolved in 2 ml Methanol. The reaction mixture was heated for 15 min at 160° C. in a microwave reactor. The solvent was removed under reduced pressure and the crude product was purified by reversed phase HPLC (AtlantisAQ) to yield 3 mg of the desired product as a white solid (71%).

1H-NMR (500 MHz, CD₃OD) 7.19 (t, 1H, J=7.9 Hz); 6.82 (d, 1H, J=8.1 Hz); 6.78 (t, 1H, J=2.0 Hz); 6.74 (ddd, 1H, J=0.8 Hz, J=2.4 Hz, J=8.1 Hz); 5.27 (s, 1H); 4.85 (m, 2H)

HRMS (M+H⁺, calc. 263.0490, found 263.0493)

### Example 17

### 4-(3-Hydroxy-phenyl)-2-thioxo-1,2,3,4,6,7-hexahydro-pyrrolo[3,4-d]pyrimidin-5-one

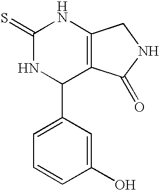

12 mg of the deprotected amine were dissolved in 2 ml Methanol. The reaction mixture was heated for 15 min at 160° C. in a microwave reactor. The solvent was removed under reduced pressure and the crude product was purified by reversed phase HPLC (AtlantisAQ) to yield 8 mg of the desired product as a colorless oil (77%).

1H-NMR (500 MHz, CD₃OD); 7.16 (t, 1H, J=7.9 Hz); 6.82 (d, 1H, J=7.1 Hz); 6.78 (m, 1H); 6.71 (ddd, 1H, J=0.8 Hz, J=2.4 Hz, J=8.1 Hz); 5.27 (s, 1H); 4.08 (d, 1H, J=19.1 Hz); 4.02 (d, 1H, J=19.1 Hz)

HRMS (M+H⁺, calc. 262.0650, found 262.0653)

### Example 18

### 4-(3-Hydroxy-phenyl)-2-thioxo-2,3,4,6,7,8-hexahydro-1H-pyrido[4,3-d]pyrimidin-5-one

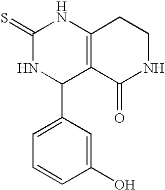

12 mg of the deprotected amine and 10 ul Triethylamine were dissolved in 500 ul Methanol. The reaction mixture was heated for 16 h at 60° C. The solvent was removed under reduced pressure and the crude product was purified on silica (10-30% Methanol in Methylene Chloride) to yield 8 mg of a white solid (80%).

1H-NMR (500 MHz, d6-DMSO); 10.34 (s, 1H); 9.41 (s, 2H); 7.19 (s, 1H); 7.11 (t, 1H, J=7.7 Hz); 6.68 (m, 2H); 6.63 (ddd, 1H, J=0.9 Hz, J=2.4 Hz, J=8.1 Hz); 5.12 (d, 1H, J=3.1 Hz); 3.18 (m, 2H); 2.51 (m, 1H); 2.42 (td, 1H, J=4.6 Hz, J=17.2 Hz)

HRMS (M+H⁺, calc. 276.0806, found 276.0808)

1-b) PM3 calculations

The PM3 calculations where performed with CAChe 6.1.1 (Fujitsu—http://www.cachesoftware.com) using default PM3 settings.

1-c) Biological Data

### Example 19

### Measuring Activity of Eg5 Inhibitors in Pure Protein Assay

We utilized a colorimetric phosphate detection assay (Funk, 2004) to measure microtubule-stimulated ATP hydrolysis by the Eg5 motor domain construct Eg5-367H (Maliga, 2002) as a function of drug concentration. Stocks solutions of each compound were prepared by serial dilution into DMSO and 0.1 microliters was added by pin transfer into KC25 buffer (20 mM PIPES-KOH, pH 6.9, 25 mM KCl, 2 mM MgC12) supplemented with 1 mM ATP-KOH, pH 6.9. KC25 supplemented with 10 micromolar taxol, 50 nanomolar Eg5-367H, and 1 micromolar taxol-polymerized microtubules was then added and the reaction incubated at 25 degrees for 2 hours. Malachite green assay solution (7% (w/v) perchloric acid, 0.0075% (w/v) malachite green oxalate, 0.2% (w/v) sodium molybdate dihydrate) was added to each reaction and the optical absorbance at 640 nm (640) was measured after a 30 minute incubation using a Wallac II automated plate reader. A640 as a function of! drug concentration (C) was fit to the equation A640=A0+B/(1+C/IC50) using Kaleidegraph 3.0, A0 is the basal A640, B is the maximum increase in A640, and IC50 is the drug concentration that inhibits half the Eg5 activity. We divided the IC50 of each drug by that of monastrol to obtain the relative potency.

Certain inventive compounds, and related acyclic intermediates, were tested. In general, compounds falling within the scope of the invention showed activity, while acyclic intermediates were inactive. Table 1 below summarizes assay results for certain compounds of the invention:

TABLE 1

| Entry | Compound | IC₅₀ (μM) |
|---|---|---|
| 1 | 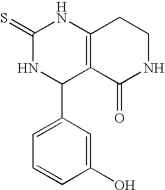 | 1.5 |
| 2 | 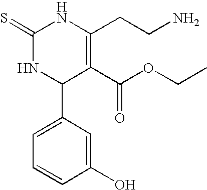 | — |
| 3 | 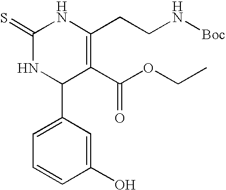 | — |
| 4 | 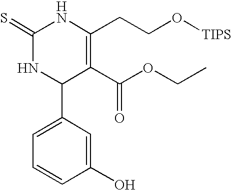 | — |
| 5 | 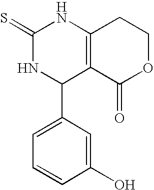 | 0.5 |
| 6 | 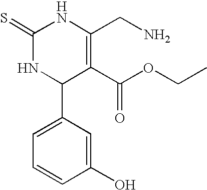 | 12 |
| 7 | 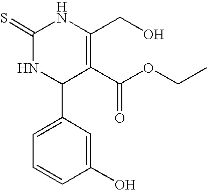 | 30 |
| 8 | 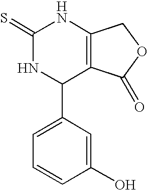 | 20 |
| 9 | 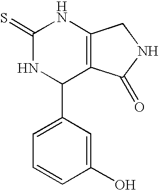 | 12 |
| 10 | 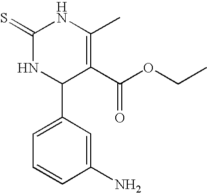 | — |
| 11 | Monastrol | 12 |

Legend: in Table 1 above, “—” means that the compound was inactive under the assay conditions used.

### Example 20

### NCI Screen

Compounds of the invention were screened against NCI's 60 cancer cell line pannel. Experimental details may be found at dtp.nci.nih.gov/branches/btb/ivclsp.html (See also Appendix B).

The two compounds tested against NCI's 60 cancer cell line pannel (MAZ1202 and MAZ1193) did not show very dominant selectivity for a specific cancer cell line. In general IC50 values were in the order of 10 PM.

CNS cancer cell lines seem to be the most sensitive with IC50 values in the range of 1-10 μM whereas leukemia cell lines exhibited the lowest sensitivity. All cell lines but one (NCI-H226) responded to the treatment.

### Example 21

### Tissue Culture Cell Assay

Hela cells were grown (Maliga, et. al. 2001) to 20% confluence in 384-well, clear-bottomed, black-walled NUNC plates and serial dilutions of prospective Eg5 inhibitors were added by pin transfer, as above [0277], incubated at 37° C. for 20 hours, fixed with TBS-0.1% Triton X100 supplemented with 4% formaldehyde, and stained stained for DNA, phosphorylated Ser10-Histone 3, and alpha-tubulin. Each drug concentration was inspected for the characteristic monoaster phenotype: condensed phospho-S10 positive chromosomes arranged in a rosette pattern around a circularly symmetric array of microtubules, and the absence of bi-polar mitotic spindles.

Certain inventive compounds, and related acyclic intermediates, were tested. In general, compounds falling within the scope of the invention showed activity, while acyclic intermediates were inactive. Table 2 below summarizes assay results for certain compounds of the invention:

TABLE 2

| Entry | Compound | IC₅₀ (μM) |
|---|---|---|
| 1 | 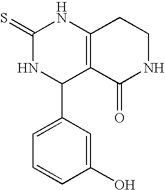 | 50 |
| 2 | 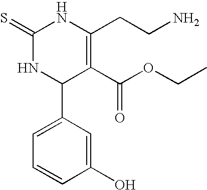 | — |
| 3 | 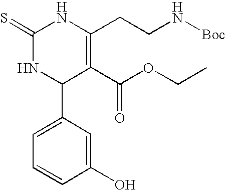 | — |
| 4 | 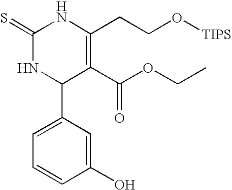 | — |
| 5 | 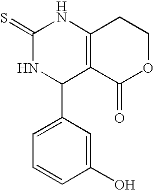 | 30 |
| 6 | 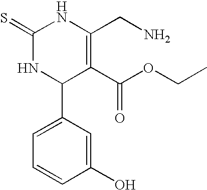 | 200 |
| 7 | 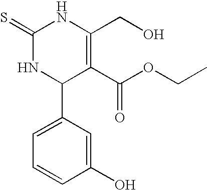 | 300 |
| 8 | 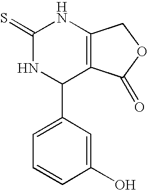 | 300 |
| 9 | 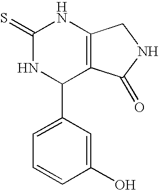 | — |
| 10 | 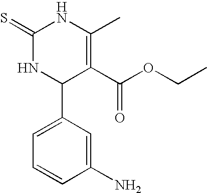 | — |
| 11 | Monastrol | 300 |

Legend: in Table 2 above, “—” means that the compound was inactive under the assay conditions used.

### Example 22

### Further Biological Characterization

Pharmacological properties of the Eg5 inhibitors of this invention may be evaluated in a number of pharmacological assays, such as those described below.

Cell Culture

Cell lines are maintained in RPMI-1640 plus 10% fetal bovine serum. Human cell lines used in one or more of the following assays described below include but are not limited to A2780 ovarian carcinoma, HCT1 16, colorectal carcinoma; HT-29, colorectal adenocarcinoma; SK-BR-3, mammary adenocarcinoma; Saos-2, osteosacroma; PC-3, prostate adenocarcinoma; and LX-1, lung carcinoma. The kangaroo rat kidney epitheilal cell line, PTK2, may also be used.

72-Hour Proliferation Assay

Cells are plated at a density of about 3,000-6,000 cells/well, depending upon the cell line used, in a 96-well plate. The cultures are grown overnight. Cells are then treated in triplicate with a seven concentration dose-response curve. The maximum concentration of DMSO may not exceed 0.5%. Cells are exposed to compound for about 72 hours. Proliferation is measured using XTT or MTS from Promega.

Clonogenic Growth Assay

Colony growth inhibition is measured using a standard clonogenic assay. Briefly, about 200 cells/well are seeded into 6-well tissue culture plates (Falcon, Franklin Lakes, N.J.) and allowed to attach for 18 hours. Assay medium consists of RPMI-1640 plus 10% fetal bovine serum. Cells are then treated in duplicate with a six concentration dose-response curve. The maximum concentration of DMSO may not exceed 0.25%. Cells are exposed to compound for about 4 to 24 hours. Compound is then removed and the cells are washed with 2 volumes of PBS. The normal growth medium is then replaced. Colonies are fed with fresh media every third day. Colony number is scored on day 10-14 using a Optimax imaging station. The compound concentration required to inhibit 50% or 90% of colony formation (IC50 or IC90, respectively) is determined by non-linear regression analysis.

Combination Studies—Clonogenic Growth Assays

Combination studies to examine the use of the Eg5 inhibitors of the present invention in combination with other antineoplastic agents may be conducted essentially in a similar fashion as standard colony growth assay with the exception of compound treatment. In the combination studies, the cells are treated with both a compound of formula land another antineoplastic agent. The compounds are administered simultaneously or sequentially; both the order of sequence and length of treatment (about 1 to 24 hours) are varied. Data evaluation is based upon the isobologram analysis and the envelope of additivity, using the line of multiplicity which compares the survival fractions of combination treatments with those of single drug treatments.

Cell Cycle Analysis

The cell cycle profile of cells treated with compounds of the present invention may be monitored by flow cytometry. Briefly, cells are seeded at a density of about 2×10⁵ per well in standard 6 well culture plates and permitted to grow for about 17 hours. Cells are then exposed to compounds of the present invention at varying concentrations for about 2 to 24 hours. Following exposure, cell populations are harvested, stained with propidium iodide to determine DNA content and also stained with the appropriate immunological reagent for protein biomarkers of mitosis and apoptosis, including, but not limited to, for example, anti-phospho-ThreonineProline, anti-M Phase Phospoprotein 2 (MMP2), and anti-p85 PARP.

Immunocytochemistry Assays

Cells are plated at a density of 200 to 2000 cells per well in 4 chamber glass slides and allowed to attach overnight. Cells are then treated with compounds of the present invention at concentrations of 100 nM to 50 μM for about 4 to 30 hours, fixed and permeabilized for subsequent staining. Stain reagents included, for example, propidium iodide, DAPI, rhodamine phalloidin, anti-αtubulin, anti-βtubulin, anti-γtubulin, and the appropriate fluorescent-tagged secondary antibodies. Cells are imaged by fluorescent and confocal fluorescent microscropy.

Preparation of Recombinant Human Eg5 Kinesin (Eg5-405)

DNA encoding full length human Eg5 kinesin is amplified by the polymerase chain reaction (PCR) using Vent DNA polymerase (NE Biolabs, Beverly, Mass.) and subcloned into an expression plasmid (pRSETa). For the PCR reaction, the template used is a pBluescript vector containing the full length coding sequence for human Eg5 (a gift from Anne Blangy). The 5′ primer (5′-GCA ACG ATT AAT ATG GCG TCG CAG CCA AAT TCG TCT GCG AAG) contains an Ase I cleavage site upstream of the Eg5 start codon. The 3′ primer (5′-GCA ACG CTC GAG TCA GTG ATG ATG GTG GTG ATG CAT GAC TCT AAA ATT TTC TTC AGA AAT) is complimentary to amino acid 405 and adds a downstream six histidine tag (6-HIS) followed by a UGA stop codon, and Xho I cleavage site.

The resulting PCR DNA amplification product and also the target plasmid to be used as the expression plasmid (pRSETa) are double digested with Ase I/Xho I and Nde I/Xho I respectively (New England Biolabs). Both products of the two restriction enzyme double digests are resolved and purified by agarose gel electrophoresis. The bands on the agarose gel corresponding to the desired DNA fragments, more than 2 kb for pRSETa and 1.2 kb for Eg5-405 are excised and purified (Qiagen Gel Purification Kit). The cleaved and purified DNA fragments are ligated together using T4 DNA ligase (New England Biolabs). The ligation products are transformed into E. coli DH5α chemically competent cells (Life Technologies), and selected by overnight growth on LB ampicillin plates. Transformants are amplified by growth of E. coli in LB ampicillin. Plasmids are purified (Qiagen Midiprep), and sequenced (Harvard Medical School Biopolymer Facilities).

Purification of Eg5-405 and K560 (560 Amino Acid Kinesin Construct):

BL21 pLysS (DE3) bacteria are transfected with the expression plasmid described in the preceding section and grown overnight at 37° C. on LB plates containing 100 ug/ml ampicillin (LB-amp). Several colonies are picked and grown at 37° C. in 1 ml LB-amp, pooled and used to inoculate each of six 1.5 L of LB-amp. These 1 L cultures are incubated at 37° C. on a shaker (200 RPM) until the optical density (O.D.) of the culture reached an absorbance of approximately A_{600 nM}=0.5 O.D. The cultures are cooled to 20° C., induced with 24 mg/ml of isopropyl beta-D-thiogalactopyranoside (IPTG; Boehringer Mannheim) and incubated at room temperature for approximately 3 hours. Cells are pelleted by centrifugation (4000×g), rinsed in phosphate buffered saline (PBS) and repelleted at 10,000×g). The bacterial pellets are flash frozen in liquid nitrogen and stored at −80° C.

The bacterial pellets are thawed on ice and resuspended in a solution containing 50 mM potassium phosphate (pH 8.0), 250 mM KCl, 0.1% Tween-20, 10 mM imidazole, 0.5 mM magnesium adenosine triphosphate (Mg-ATP), 1 mM phenylmethanesulfonyl fluoride (PMSF), and 2 mM benzimidine-HCl. To lyse the bacteria, lysozyme (1 mg/ml) and 2-mercaptoethanol (5 mM) are added to the solution to result in the indicated final concentration, incubated and sonicated (3 times seconds, repeated 3 times incubating for 1 minute on ice between each triple sonication) to break up the DNA and guarantee bacterial lysis.

The lysate is spun at 40,000×g for approximately 35 minutes at 4° C. with the resulting supernatant separated from the pellet by decanting and then incubated with a nickel-nitrilotriacetic acid resin (Ni-NTA; QIAGEN). The remaining pellet is washed three times with a wash buffer (lysis buffer supplemented with 10 mM 2-mercaptoethanol, no PMSF, and 0.1 mM Mg-ATP) to extract any remaining protein. These washes are then followed by a final wash using a low pH buffer (pH 6.0). The wash solutions are then also added to the Ni-NTA resin. The resin containing the desired tagged protein is poured into a column (Biorad, 0.8×4 cm PolyPrep Chromatography Column) and allowed to settle. The HIS-tagged proteins are eluted with a solution containing 250 mM imidazole and 150 mM KCl (pH 7.0). Protein-containing fractions of the eluate are loaded onto a Superose 6 size-exclusion column (Pharmacia) and equilibrated with a solution containing 80 mM potassium HEPES (pH 6.8), 200 mM KCl, 10 uM Mg-ATP, 1 mM dithiothreitol (DTT). Fractions containing homogeneous proteins as determined by molecular weight and mobility on SDS-PAGE are used for further enzymology experiments.

Polymerization of Microtubules:

A solution containing 1 mg/ml tubulin, 1 mM DTT, 1 mM guanosine triphosphate (GTP), 1 mM MgCl₂, 80 mM potassium HEPES (pH 6.8), and 1 mM ethylene glycol-bis(beta-aminoethyl ether)-N,N,N′N′-tetraacetic acid (EGTA) is spun at 90,000×g for 5 minutes. The solution is then warmed to 37° C. for 2 minutes. Taxol is added in stepwise as 0.01, 0.1, and 1 equivalents. The polymerization solution is placed onto a solution containing 40% glycerol, 80 mM potassium HEPES (pH 6.8), 1 mM MgCl₂, and 1 mM EGTA, and centrifuged at 90,000×g for approximately 50 minutes. The resulting microtubule pellet is washed extensively with and resuspended in resuspension buffer containing 80 mM potassium HEPES (pH 6.8), 1 mM MgCl₂, and 1 mM EGTA.

In vitro NADH Enzyme Coupled ATPase: (variation on methods described in Crevel, Lockhart, and Cross. J. Mol. Biol. (1997) 273, 160-170.)

A solution containing the microtubule resuspension buffer is supplemented with 1 mM Mg-ATP, 100 uM nicotinamide adenine dinucleotide (NADH), 1 mM phosphoenol pyruvate, 5 μg/ml pyruvate kinase, 7.5 μg/ml lactate dehydrogenase, 0.7 μM resuspended microtubules, and 0.5% dimethylsulfoxide (DMSO).

Next, serial dilutions of the test compound are prepared in the supplemented microtubule resuspension buffer. Purified recombinant human Eg5 protein is added resulting in a final concentration of 50 μM. The subsequent fluorescent reaction is measured in NUNC black walled 384 well plates at 340 nm using a Wallac plate reader.

1. An isolated compound having the structure: 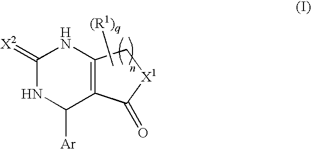
or pharmaceutically acceptable derivative thereof;
wherein Ar is an aromatic or heteroaromatic moiety;
X¹ is O or NR^{X1}, wherein R^{X1} is hydrogen or an aliphatic, heteroaliphatic, aromatic or heteroaromatic moiety;
X² is O or S;
n is 1 or 2;
q is an integer from 0-4; and
each occurrence of R₁ is independently hydrogen, halogen, hydroxyl, or an aliphatic, heteroaliphatic, aromatic or heteroaromatic moiety.

2. The compound of claim 1 having the structure:

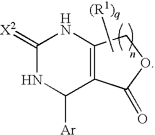

3. The compound of claim 1 having the structure:

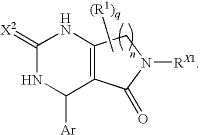

4. The compound of claim 1 having the structure:

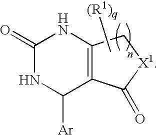

5. The compound of claim 1 having the structure:

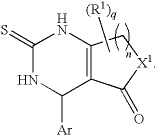

6. The compound of claim 1 having the structure: 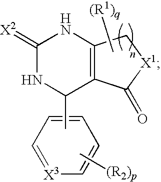
wherein X³ is N or CR²; p is 0-5; and each occurrence of R² is independently hydrogen, halogen, CN, NO₂, alkyl, alkenyl, alkynyl, heteroalkyl, heteroalkenyl, heteroalkynyl, cycloalkyl, heterocycle, aryl, heteroaryl, -(alkyl)cycloalkyl, -(alkyl)heterocycle, -(heteroalkyl)cycloalkyl, -(heteroalkyl)heterocycle, -(alkyl)aryl, -(heteroalkyl)aryl, -(alkyl)heteroaryl, -(heteroalkyl)heteroaryl, —OR^{2A}, —S(═O)ₓR^{2A}, —N(R^{2A})₂, —SO₂N(R^{2A})₂, C(═O)R^{2A}, —C(═O)N(R^{2A})₂, —C(═O)OR^{2A}, —N(R^{2A})C(═O)R^{2B} or —N(R^{2A})SO₂R^{2B}, wherein x is 0, 1 or 2; and each occurrence of R^{2A} and R^{2B} is independently hydrogen, lower alkyl, lower heteroalkyl, aryl, heteroaryl, -(alkyl)aryl or -(alkyl)heteroaryl; or R^{2A} and R^{2B}, taken together with the atoms to which they are attached, form a substituted or unsubstituted 5-8 membered heterocyclic moiety.

7. The compound of claim 1 having the structure: 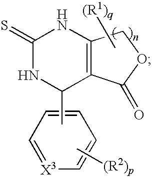
wherein p is 0-5; and each occurrence of R² is independently hydrogen, halogen, CN, NO₂, alkyl, alkenyl, alkynyl, heteroalkyl, heteroalkenyl, heteroalkynyl, cycloalkyl, heterocycle, aryl, heteroaryl, -(alkyl)cycloalkyl, -(alkyl)heterocycle, -(heteroalkyl)cycloalkyl, -(heteroalkyl)heterocycle, -(alkyl)aryl, -(heteroalkyl)aryl, -(alkyl)heteroaryl , -(heteroalkyl)heteroaryl, —OR^{2A}, —S(═O)ₓR^{2A}, —N(R^{2A})₂, —SO₂N(R^{2A})₂, —C(═O)R^{2A}, —C(═O)N(R^{2A})₂, —C(═O)OR^{2A}, —N(R^{2A})C(═O)R^{2B} or —N(R^{2A})SO₂R^{2B}, wherein x is 0, 1 or 2; and each occurrence of R^{2A} and R^{2B} is independently hydrogen, lower alkyl, lower heteroalkyl, aryl, heteroaryl, -(alkyl)aryl or -(alkyl)heteroaryl; or R^{2A} and R^{2B}, taken together with the atoms to which they are attached, form a substituted or unsubstituted 5-8 membered heterocyclic moiety.

8. The compound of claim 7 having the structure: 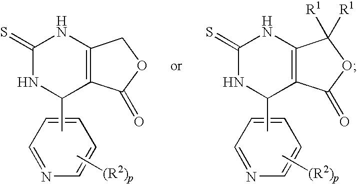
wherein each occurrence of R¹ is independently halogen or lower alkyl.

9. The compound of claim 7 having the structure: 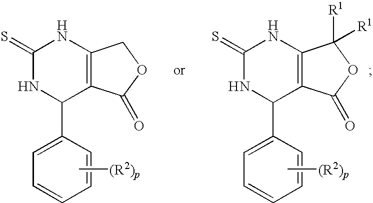
wherein each occurrence of R¹ is independently halogen or lower alkyl.

10. The compound of claim 7 having the structure: 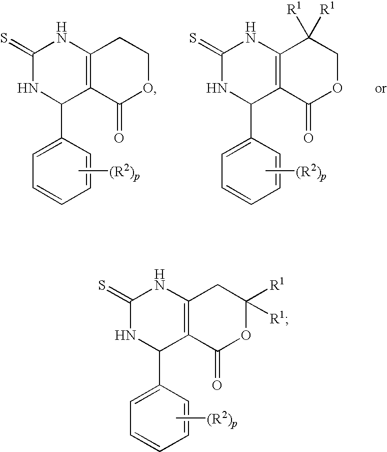
wherein each occurrence of R¹ is independently halogen or lower alkyl.

11. The compound of claim 7 having the structure: 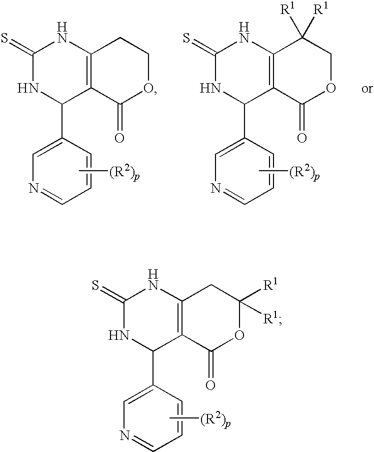
wherein each occurrence of R¹ is independently halogen or lower alkyl.

12. The compound of claim 1 having the structure: 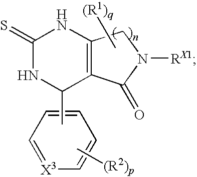
wherein R^{X1} is hydrogen, lower alkyl, acyl or a nitrogen protecting group; p is 0-5; and each occurrence of R² is independently hydrogen, halogen, CN, NO₂, alkyl, alkenyl, alkynyl, heteroalkyl, heteroalkenyl, heteroalkynyl, cycloalkyl, heterocycle, aryl, heteroaryl, -(alkyl)cycloalkyl, -(alkyl)heterocycle, -(heteroalkyl)cycloalkyl, -(heteroalkyl)heterocycle, -(alkyl)aryl, -(heteroalkyl)aryl, -(alkyl)heteroaryl , -(heteroalkyl)heteroaryl, —OR^{2A}, —S(═O)ₓR^{2A}, —N(R^{2A})₂, —SO₂N(R^{2A})₂, —C(═O)R^{2A}, —C(═O)N(R^{2A})₂, —C(═O)OR^{2A}, —N(R^{2A})C(═O)R^{2B} or —N(R^{2A})SO₂R^{2B}, wherein x is 0, 1 or 2; and each occurrence of R^{2A} and R^{2B} is independently hydrogen, lower alkyl, lower heteroalkyl, aryl, heteroaryl, -(alkyl)aryl or -(alkyl)heteroaryl; or R^{2A} and R^{2B}, taken together with the atoms to which they are attached, form a substituted or unsubstituted 5-8 membered heterocyclic moiety.

13. The compound of claim 12 having the structure: 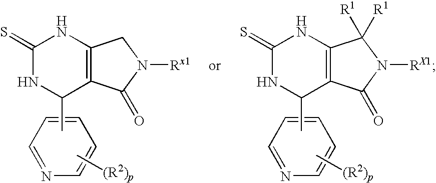
wherein each occurrence of R¹ is independently halogen or lower alkyl.

14. The compound of claim 12 having the structure: 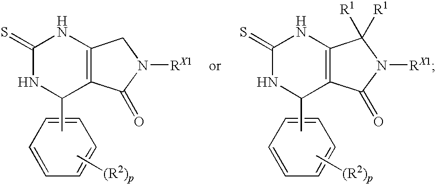
wherein each occurrence of R¹ is independently halogen or lower alkyl.

15. The compound of claim 12 having the structure: 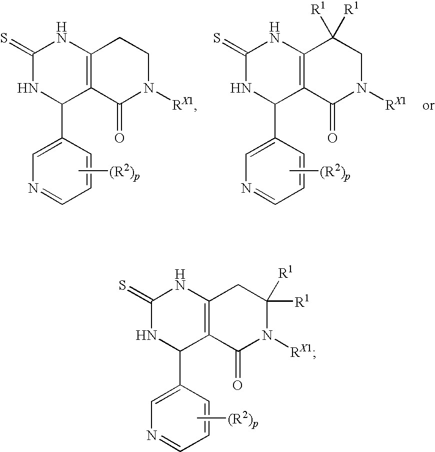
wherein each occurrence of R¹ is independently halogen or lower alkyl.

16. The compound of claim 12 having the structure: 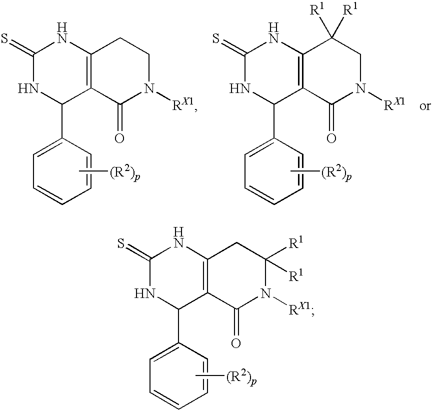
wherein each occurrence of R¹ is independently halogen or lower alkyl.

17. The compound of claim 1 having the structure: 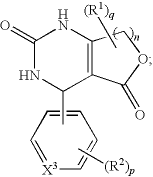
wherein p is 0-5; and each occurrence of R² is independently hydrogen, halogen, CN, NO₂, alkyl, alkenyl, alkynyl, heteroalkyl, heteroalkenyl, heteroalkynyl, cycloalkyl, heterocycle, aryl, heteroaryl, -(alkyl)cycloalkyl, -(alkyl)heterocycle, -(heteroalkyl)cycloalkyl, -(heteroalkyl)heterocycle, -(alkyl)aryl, -(heteroalkyl)aryl, -(alkyl)heteroaryl , -(heteroalkyl)heteroaryl, —OR^{2A}, —S(═O)ₓR^{2A}, —N(R^{2A})₂, —SO₂N(R^{2A})₂, —C(═O)R^{2A}, —C(═O)N(R^{2A})₂, —C(═O)OR^{2A}, —N(R^{2A})C(═O)R^{2B} or —N(R^{2A})SO₂R^{2B}, wherein x is 0, 1 or 2; and each occurrence of R^{2A} and R^{2B} is independently hydrogen, lower alkyl, lower heteroalkyl, aryl, heteroaryl, -(alkyl)aryl or -(alkyl)heteroaryl; or R^{2A} and R^{2B}, taken together with the atoms to which they are attached, form a substituted or unsubstituted 5-8 membered heterocyclic moiety.

18. The compound of claim 17 having the structure: 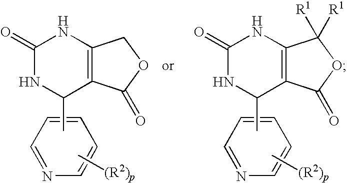
wherein each occurrence of R¹ is independently halogen or lower alkyl.

19. The compound of claim 17 having the structure: 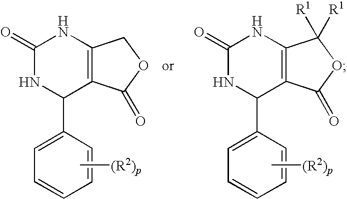
wherein each occurrence of R¹ is independently halogen or lower alkyl.

20. The compound of claim 17 having the structure: 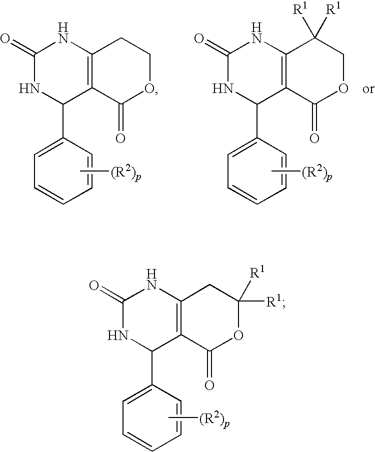
wherein each occurrence of R¹ is independently halogen or lower alkyl.

21. The compound of claim 17 having the structure: 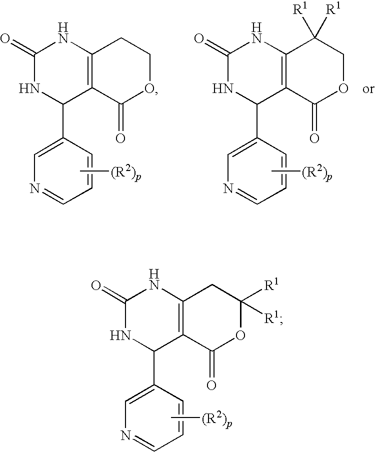
wherein each occurrence of R¹ is independently halogen or lower alkyl.

22. The compound of claim 1 having the structure: 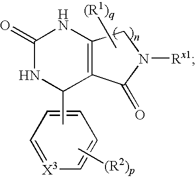
wherein R^{X1} is hydrogen, lower alkyl, acyl or a nitrogen protecting group; p is 0-5; and each occurrence of R² is independently hydrogen, halogen, CN, NO₂, alkyl, alkenyl, alkynyl, heteroalkyl, heteroalkenyl, heteroalkynyl, cycloalkyl, heterocycle, aryl, heteroaryl, -(alkyl)cycloalkyl, -(alkyl)heterocycle, -(heteroalkyl)cycloalkyl, -(heteroalkyl)heterocycle, -(alkyl)aryl, -(heteroalkyl)aryl, -(alkyl)heteroaryl , -(heteroalkyl)heteroaryl, —OR^{2A}, —S(═O)ₓR^{2A}, —N(R^{2A})₂, —SO₂N(R^{2A})₂, —C(═O)R^{2A}, —C(═O)N(R^{2A})₂, —C(═O)OR^{2A}, —N(R^{2A})C(═O)R² or —N(R^{2A})SO₂R^{2B}, wherein x is 0, 1 or 2; and each occurrence of R^{2A} and R^{2B} is independently hydrogen, lower alkyl, lower heteroalkyl, aryl, heteroaryl, -(alkyl)aryl or -(alkyl)heteroaryl; or R^{2A} and R^{2B}, taken together with the atoms to which they are attached, form a substituted or unsubstituted 5-8 membered heterocyclic moiety.

23. The compound of claim 22 having the structure: 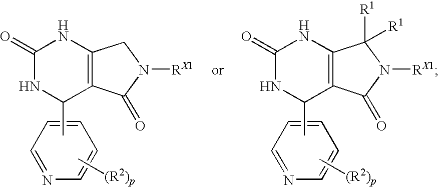
wherein each occurrence of R¹ is independently halogen or lower alkyl.

24. The compound of claim 22 having the structure: 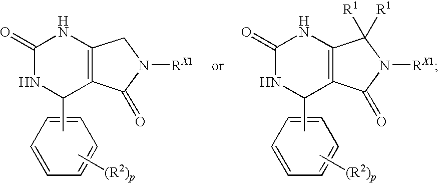
wherein each occurrence of R¹ is independently halogen or lower alkyl.

25. The compound of claim 22 having the structure: 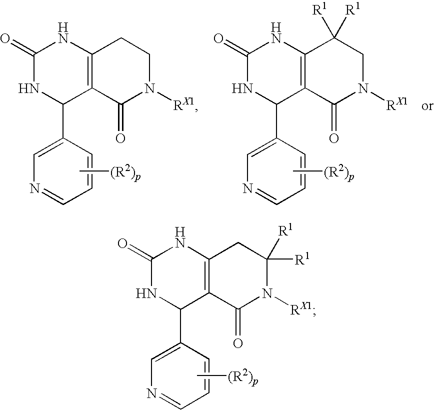
wherein each occurrence of R¹ is independently halogen or lower alkyl.

26. The compound of claim 22 having the structure: 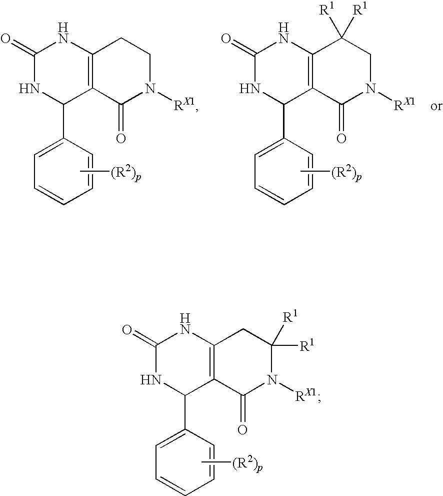
wherein each occurrence of R¹ is independently halogen or lower alkyl.

27. The compound of claim 1, wherein X¹ is O.

28. The compound of claim 1, wherein X¹ is NR^{X1}, wherein R^{X1} is hydrogen, alkyl, alkenyl, alkynyl, heteroalkyl, heteroalkenyl, heteroalkynyl, cycloalkyl, heterocycle, acyl, aryl, heteroaryl, -(alkyl)cycloalkyl, -(alkyl)heterocycle, -(heteroalkyl)cycloalkyl, -(heteroalkyl)heterocycle, -(alkyl)aryl, -(heteroalkyl)aryl, -(alkyl)heteroaryl or -(heteroalkyl)heteroaryl.

29. The compound of claim 1, wherein X¹ is NR^{X1}, wherein R^{X1} is hydrogen, lower alkyl, lower heteroalkyl, cycloalkyl, heterocycle, lower acyl, aryl, heteroaryl, -(alkyl)cycloalkyl, -(alkyl)heterocycle, -(alkyl)aryl, -(heteroalkyl)aryl or -(alkyl)heteroaryl.

30. The compound of claim 1 wherein X¹ is NR^{X1}, wherein R^{X1} is hydrogen, lower alkyl, lower acyl, aryl or heteroaryl.

31. The compound of claim 1, wherein X¹ is NR^{X1} wherein R^{X1} is hydrogen, lower alkyl or lower acyl.

32. The compound of claim 1, wherein X² is O.

33. The compound of claim 1, wherein X² is S.

34. The compound of claim 1 wherein n is 1.

35. The compound of claim 1 wherein n is 2.

36. The compound of claim 6 wherein p is 1.

37. The compound of claim 1 wherein q is 0.

38. The compound of claim 1 wherein q is 1.

39. The compound of claim 1 wherein n is 1 and q is 0.

40. The compound of claim 1 wherein n is 2 and q is 0.

41. The compound of claim 1 wherein each occurrence of R¹ is independently hydrogen, halogen, hydroxy, alkyl, alkenyl, alkynyl, heteroalkyl, heteroalkenyl, heteroalkynyl, cycloalkyl, heterocycle, aryl, heteroaryl, -(alkyl)cycloalkyl, -(alkyl)heterocycle, -(heteroalkyl)cycloalkyl, -(heteroalkyl)heterocycle, -(alkyl)aryl, -(heteroalkyl)aryl, -(alkyl)heteroaryl, -(heteroalkyl)heteroaryl, —OR^{1A}, —SR^{1A}, N(R^{1A})₂, —SO₂N(R^{1A})₂, —C(═O)N(R^{1A})₂, halogen, —CN, —NO₂, —C(═O)R^{1A}, —C(═O)OR^{1A}, —N(R^{1A})C(═O)R^{1B} or —N(R^{1A})SO₂R^{1B}, wherein each occurrence of R^{1A} and R^{1B} is independently hydrogen, lower alkyl, lower heteroalkyl, aryl, heteroaryl, -(alkyl)aryl or -(alkyl)heteroaryl; or R^{1A} and R^{1B}, taken together with the atoms to which they are attached, form a substituted or unsubstituted 5-8 membered heterocyclic moiety.

42. The compound of claim 1 wherein each occurrence of R¹ is independently hydrogen, halogen, hydroxy, lower alkyl, lower heteroalkyl, 3-6 membered cycloalkyl, 3-6 membered heterocycle, aryl, heteroaryl, -(lower alkyl)cycloalkyl, -(lower alkyl)heterocycle, -(lower alkyl)aryl or -(lower alkyl)heteroaryl.

43. The compound of claim 1 wherein R¹, for each occurrence, is hydrogen.

44. The compound of claim 6 wherein each occurrence of R² is independently hydrogen, halogen, CN, NO₂, alkyl, alkenyl, alkynyl, heteroalkyl, heteroalkenyl, heteroalkynyl, cycloalkyl, heterocycle, aryl, heteroaryl, -(alkyl)cycloalkyl, -(alkyl)heterocycle, -(heteroalkyl)cycloalkyl, -(heteroalkyl)heterocycle, -(alkyl)aryl, -(heteroalkyl)aryl, -(alkyl)heteroaryl , -(heteroalkyl)heteroaryl, —OR^{2A}, —S(═O)ₓR^{2A}, —N(R^{2A})₂, —SO₂N(R^{2A})₂, —C(═O)R^{2A}, —C(═O)N(R^{2A})₂, —C(═O)OR^{2A}, —N(R^{2A})C(═O)R² or —N(R^{2A})SO₂R^{2B}, wherein x is 0, 1 or 2; and each occurrence of R^{2A} and R^{2B} is independently hydrogen, lower alkyl, lower heteroalkyl, aryl, heteroaryl, -(alkyl)aryl or -(alkyl)heteroaryl; or R^{2A} and R^{2B}, taken together with the atoms to which they are attached, form a substituted or unsubstituted 5-8 membered heterocyclic moiety.

45. The compound of claim 6 wherein each occurrence of R² is independently hydrogen, halogen, CN, NO₂, lower alkyl, lower heteroalkyl, 3-6 membered cycloalkyl, 3-8 membered heterocycle, aryl, heteroaryl, -(lower alkyl)cycloalkyl, -(lower alkyl)heterocycle, -(lower heteroalkyl)cycloalkyl, -(lower heteroalkyl)heterocycle, -(lower alkyl)aryl, -(lower heteroalkyl)aryl, -(lower alkyl)heteroaryl , -(lower heteroalkyl)heteroaryl, —OR^{2A}, —SR^{2A}, —N(R^{2A})₂, —C(═O)R^{2A}, —C(═O)N(R^{2A})₂, —C(═O)OR^{2A}, —N(R^{2A})C(═O)R^{2B} or —N(R^{2A})SO₂R^{2B}, wherein each occurrence of R^{2A} and R^{2B} is independently hydrogen, lower alkyl, lower heteroalkyl, aryl, heteroaryl, -(lower alkyl)aryl or -(lower alkyl)heteroaryl; or R^{2A} and R^{2B}, taken together with the atoms to which they are attached, form a substituted or unsubstituted 5-8 membered heterocyclic moiety.

46. The compound of claim 6 wherein each occurrence of R² is independently hydrogen, halogen, lower alkyl, lower heteroalkyl, —OR^{2A}, —SR^{2A}, —N(R^{2A})₂, —C(═O)R^{2A}, —C(═O)N(R^{2A})₂, —C(═O)OR^{2A}, —N(R^{2A})C(═O)R^{2B} or —N(R^{2A})SO₂R^{2B}, wherein each occurrence of R^{2A} and R^{2B} is independently hydrogen, lower alkyl, lower heteroalkyl, aryl, heteroaryl, -(lower alkyl)aryl or -(lower alkyl)heteroaryl; or R^{2A} and R^{2B}, taken together with the atoms to which they are attached, form a substituted or unsubstituted 5-8 membered heterocyclic moiety.

47. The compound of claim 6 wherein each occurrence of R² is independently hydrogen, halogen, lower alkyl or —OR^{2A}; wherein R^{2A} is hydrogen, lower alkyl, aryl, heteroaryl, -(lower alkyl)aryl or -(lower alkyl)heteroaryl.

48. The compound of claim 6 wherein each occurrence of R² is independently hydrogen, halogen, lower alkyl or —OR^{2A}; wherein R^{2A} is hydrogen, or lower alkyl.

49. The compound of claim 6 wherein each occurrence of R² is independently hydrogen, hydroxyl or lower alkoxy.

50. The compound of claim 1 wherein Ar is substituted or unsubstituted phenyl or naphthyl, or a moiety having one of the following structures: 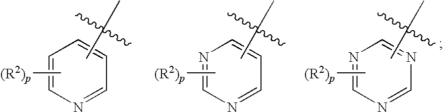
wherein p is 0-5; and each occurrence of R² is independently hydrogen, halogen, CN, NO₂, alkyl, alkenyl, alkynyl, heteroalkyl, heteroalkenyl, heteroalkynyl, cycloalkyl, heterocycle, aryl, heteroaryl, -(alkyl)cycloalkyl, -(alkyl)heterocycle, -(heteroalkyl)cycloalkyl, -(heteroalkyl)heterocycle, -(alkyl)aryl, -(heteroalkyl)aryl, -(alkyl)heteroaryl , -(heteroalkyl)heteroaryl, —OR^{2A}, —S(═O)ₓR^{2A}, —N(R^{2A})₂, —SO₂N(R^{2A})₂, —C(═O)R^{2A}, —C(═O)N(R^{2A})₂, —C(═O)OR^{2A}, —N(R^{2A})C(═O)R^{2B} or —N(R^{2A})SO₂R^{2B}, wherein x is 0, 1 or 2; and each occurrence of R^{2A} and R^{2B} is independently hydrogen, lower alkyl, lower heteroalkyl, aryl, heteroaryl, -(alkyl)aryl or -(alkyl)heteroaryl; or R^{2A} and R^{2B}, taken together with the atoms to which they are attached, form a substituted or unsubstituted 5-8 membered heterocyclic moiety.

51. The compound of claim 50 wherein Ar is substituted or unsubstituted phenyl or pyridyl.

52. The compound of claim 50 wherein Ar has one of the following structures: 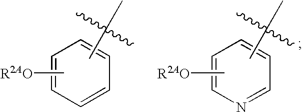
wherein R^{2A} is hydrogen, alkyl, alkenyl, alkynyl, heteroalkyl, heteroalkenyl, heteroalkynyl, cycloalkyl, heterocycle, acyl, aryl, heteroaryl, -(alkyl)cycloalkyl, -(alkyl)heterocycle, -(heteroalkyl)cycloalkyl, -(heteroalkyl)heterocycle, -(alkyl)aryl, -(heteroalkyl)aryl, -(alkyl)heteroaryl , -(heteroalkyl)heteroaryl or an oxygen protecting group.

53. The compound of claim 50 wherein Ar has one of the following structures: 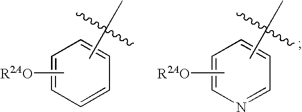
wherein R^{2A} is hydrogen, lower alkyl, lower heteroalkyl, cycloalkyl, heterocycle, lower acyl, aryl, heteroaryl, -(alkyl)cycloalkyl, -(alkyl)heterocycle, -(alkyl)aryl, -(heteroalkyl)aryl, -(alkyl)heteroaryl or an oxygen protecting group.

54. The compound of claim 50 wherein Ar has one of the following structures: 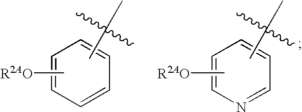
wherein R^{2A} is hydrogen, lower alkyl, lower acyl, aryl, heteroaryl or an oxygen protecting group.

55. The compound of claim 50 wherein Ar has one of the following structures: 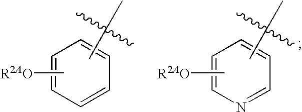
wherein R^{2A} is hydrogen, lower alkyl, lower acyl or an oxygen protecting group.

56. The compound of claim 50 wherein Ar has one of the following structures:

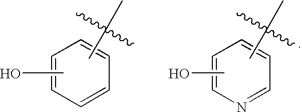

57. The compound of claim 50 wherein Ar has the structure: 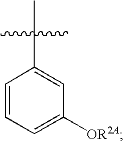
wherein R^{2A} is hydrogen, alkyl, alkenyl, alkynyl, heteroalkyl, heteroalkenyl, heteroalkynyl, cycloalkyl, heterocycle, acyl, aryl, heteroaryl, -(alkyl)cycloalkyl, -(alkyl)heterocycle, -(heteroalkyl)cycloalkyl, -(heteroalkyl)heterocycle, -(alkyl)aryl, -(heteroalkyl)aryl, -(alkyl)heteroaryl , -(heteroalkyl)heteroaryl or an oxygen protecting group.

58. The compound of claim 50 wherein Ar has the structure: 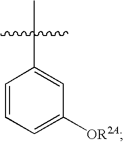
wherein R^{2A} is hydrogen, lower alkyl, lower heteroalkyl, cycloalkyl, heterocycle, lower acyl, aryl, heteroaryl, -(alkyl)cycloalkyl, -(alkyl)heterocycle, -(alkyl)aryl, -(heteroalkyl)aryl, -(alkyl)heteroaryl or an oxygen protecting group.

59. The compound of claim 50 wherein Ar has the structure: 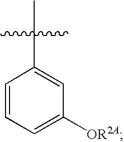
wherein R^{2A} is hydrogen, lower alkyl, lower acyl, aryl, heteroaryl or an oxygen protecting group.

60. The compound of claim 50 wherein Ar has the structure: 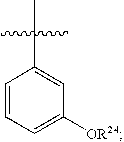
wherein R^{2A} is hydrogen, lower alkyl, lower acyl or an oxygen protecting group.

61. The compound of claim 50 wherein Ar has the structure:

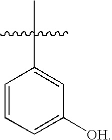

62. The compound of claim 1 wherein each occurrence of R¹ is methyl.

63. The compound of claim 1 having the structure:

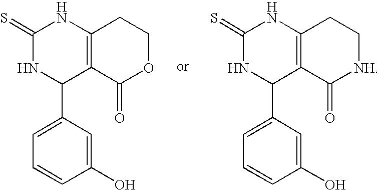

64. A pharmaceutical composition comprising at least one compound of claim 1 and further comprising a pharmaceutically acceptable carrier.

65. A method for treating or lessening the severity of a condition via modulation of Eg5 protein activity comprising administering to a subject in need thereof an effective amount of at least one compound of claim 1 and a pharmaceutically acceptable carrier.

66. The method of claim 65 wherein the condition is cancer.

67. A method for inducing mitotic arrest in cells comprising contacting the cells with an effective amount of at least one compound of claim 1 and optionally a pharmaceutically acceptable carrier.

68. A method for inducing apoptosis in cells comprising contacting the cells with an effective amount of at least one compound of claim 1 and optionally a pharmaceutically acceptable carrier.
